(19) United States
(12) Patent Application Publication
(10) Pub. No.: US 2023/0114365 A1
(43) Pub. Date: Apr. 13, 2023

(54) SYSTEMS AND METHODS FOR DISTINGUISHING PATHOLOGICAL MUTATIONS FROM CLONAL HEMATOPOIETIC MUTATIONS IN PLASMA CELL-FREE DNA BY FRAGMENT SIZE ANALYSIS

(71) Applicant: Memorial Sloan Kettering Cancer Center, New York, NY (US)

(72) Inventors: Wai Yi Tsui, New York, NY (US); Francesco Marass, New York, NY (US); Luis Diaz, JR., New York, NY (US)

(21) Appl. No.: 17/914,731

(22) PCT Filed: Mar. 18, 2021
(86) PCT No.: PCT/US2021/022921

§ 371 Date: Sep. 26, 2022

Related U.S. Application Data
(60) Provisional application No. 63/000,426, filed on Mar. 26, 2020.

(51) Int. Cl. G16B 20/00; C12Q 1/6886; G16B 40/20

(52) U.S. Cl. CPC G16B 20/00; C12Q 1/6886; G16B 40/20; C12Q 2600/158; C12Q 2600/156

(57) ABSTRACT

The genomic data processing systems and methods described herein can accurately detect mutations in nucleic acid (e.g., cell free DNA (cfDNA) sequence reads associated with plasma nucleic acid samples. The genomic data processing system of the present disclosure distinguishes mutations derived from a tumor from mutations derived of clonal hematopoietic (CH) origin. The origin of mutated DNA fragments can be more accurately determined by analyzing fragment sizes in cfDNA to generate tumor and CH regions of interest (ROIs) in corresponding size profiles. A mutation can be more accurately classified using a metric based on proportions of fragments in the ROIs.

### STATEMENT OF RELATED APPLICATIONS

This application claims priority as a PCT Application to U.S. Provisional Patent Application No. 63/000,426, filed Mar. 26, 2020, the entire contents of which is incorporated herein by reference.

### STATEMENT REGARDING FEDERALLY SPONSORED RESEARCH

This invention was made with government support under CA008748 awarded by the National Institutes of Health. The government has certain rights in the invention.

### FIELD OF THE DISCLOSURE

The present disclosure is directed to processing sequence data on cell-free DNA (cfDNA) to discriminate between mutations of clonal (e.g., clonal hematopoietic (CH)) origin and mutations of another pathological (e.g., tumor) origin and, and to identifying cancer-related mutations in cfDNA sequence data.

### BACKGROUND OF THE DISCLOSURE

The following description of the background of the present technology is provided simply as an aid in understanding the present technology and is not admitted to describe or constitute prior art to the present technology.

Tumors continually shed DNA into the circulation (circulating tumor DNA, or ctDNA), where it is readily accessible (Stroun et al., Eur J Cancer Clin Oncol 23:707-712 (1987)). Analysis of such cancer-derived cell-free DNA (cfDNA) has the potential to revolutionize cancer detection, tumor genotyping, and disease monitoring. For example, noninvasive access to tumor-derived DNA via liquid biopsies is particularly attractive for solid tumors. However, in most early- and many advanced-stage solid tumors, ctDNA blood levels are extremely low (˜0.1%) (Bettegowda, C. et al., Sci. Transl. Med. 6:224ra24 (2014); Newman, A. M. et al., Nat. Med. 20:548-554 (2014)), thus complicating ctDNA detection and analysis. Mutation fractions in cfDNA are often lower than those observed in tissue samples from the same subject and may approach the noise levels of next-generation sequencing workflows, making it impossible to distinguish true somatic mutations from artifacts. Recovery of cfDNA molecules and non-biological errors introduced during library preparation and sequencing limit analytical sensitivity and continue to represent a major obstacle for ultrasensitive ctDNA profiling.

Noninvasive detection of somatic, solid tumor-derived mutations in the blood is an important clinical and investigative tool. However, analysis of cfDNA for somatic mutations can be confounded by the presence of mutations that are not of tumor origin. These include germline alterations, mutations from clonal events in non-neoplastic tissue, and artifacts from the sequencing process (Ptashkin et al., JAMA Oncology 2018; 4:1589-93). The most abundant set of clonal mutations are derived from the hematopoietic system (“clonal hematopoiesis” (CH) mutations) and may be mistaken for tumor mutations since similar genetic alterations may be present in both (Bauml & Levy, Clin Cancer Res 2018; 24:4352-4).

### SUMMARY

The present disclosure provides methods and systems for analyzing size profiles of cfDNA fragments bearing mutations to discriminate between mutations of clonal hematopoietic origin and mutations of another pathological origin (e.g., tumor). The present disclosure is related to more sensitive and high-throughput systems and methods for effective detection of somatic mutations from cfDNA, such as for early-stage cancer subjects.

In one aspect, embodiments of the disclosure relate to a computer-implemented method. The method may be for distinguishing clonal hematopoietic derived mutations and other pathogenic (e.g., tumor-derived) mutations in cell-free DNA (cfDNA). The method may comprise: acquiring, by one or more processors, from a sequencing device, sequence reads corresponding to cfDNA fragments in a sample (e.g., plasma or serum) of a test subject; detecting, by the one or more processors, using the sequence reads corresponding to the cfDNA fragments, a gene mutation in the cfDNA; generating, by the one or more processors, a size profile for a set of cfDNA fragments with the gene mutation of specific origins, the size profile identifying how many cfDNA fragments are detected for each fragment length in a plurality of fragment lengths; classifying, by the one or more processors, in the set of cfDNA fragments, a first subset of cfDNA fragments as having a tumor origin and a second subset of cfDNA fragments as having a CH origin by feeding the size profile as an input to a mutation-specific predictive model that is configured to generate a first set of one or more ranges of fragment lengths for fragments with the tumor origin and a second set of one or more ranges of fragment lengths for fragments of the CH origin, wherein the first subset of cfDNA fragments have lengths falling in the first set of ranges and the second subset of cfDNA fragments have lengths falling in the second set of ranges; and generating, by the one or more processors, a characterization of the mutation based on the classifying of cfDNA fragments.

In various embodiments, the method may further comprise generating a metric (or multiple metrics) based on the size profile. Generating the characterization may comprise identifying an origin of the gene mutation based on a comparison of the metric (or multiple metrics) with a metric threshold. The metrics may include a proportion of fragments in one of the subsets of cfDNA fragments to fragments in both of the subsets of cfDNA fragments. The predictive model may be further configured to generate the metric threshold.

In various embodiments, the method may comprise training the predictive model by: acquiring, by the one or more processors, from the sequencing device, sequence reads corresponding to cfDNA fragments in samples of a plurality of subjects with known tumor mutations and/or known CH mutations; and generating, by the one or more processors, using the sequence reads from the sequencing device, a tumor fragment size profile and a CH fragment size profile. The method may further comprise training the predictive model by: applying, by the one or more processors, a smoothing operation to the size profiles to obtain a trend line; and defining, by the one or more processors, one or more tumor regions of interest (ROIs) and one or more CH ROIs in the trend line. The tumor ROI may correspond with the first set of ranges of fragment lengths and the CH ROI may correspond with the second set of ranges of fragment lengths. Fragment lengths in the first set of ranges may be larger than fragment lengths in the second set of ranges, or fragment lengths in the first set of ranges may be smaller than fragment lengths in the second set of ranges.

In various embodiments, the method may comprise determining a difference between the tumor fragment size profile and the CH fragment size profile, wherein the smoothing operation is applied to the difference to obtain the trend line.

In various embodiments, the predictive model may further be trained, by the one or more processors, for each mutation, using a metric based on the proportion of fragments in the tumor and CH ROIs. In various embodiments, the metric for each mutation may be a number of cfDNA fragments with lengths in the one or more tumor ROIs divided by a total number of cfDNA fragments with lengths in both the one or more tumor ROIs and the one or more CH ROIs. In various embodiments, the metric for each mutation may be a number of cfDNA fragments with lengths in the one or more CH ROIs divided by a total number of cfDNA fragments with lengths in both the one or more tumor ROIs and the one or more CH ROIs.

In various embodiments, the method may comprise selecting a metric threshold for use in classifying cfDNA fragments as having the tumor-derived mutation or the CH-derived mutation.

In various embodiments, the predictive model may be based on a tumor fragment size profile and a CH fragment size profile.

In various embodiments, the trend line may include a set of features, and each tumor ROI and CH ROI may be defined according to one of the features in the set of features. In example embodiments, the set of features may be a set of extrema (e.g., maximums and/or minimums), which may comprise a first extremum and a second extremum. The tumor ROI may comprise the first extremum, and the CH ROI may comprise the second extremum. For example, the tumor ROI may be centered about (or terminating in or otherwise comprising) the first extremum, and the CH ROI may be centered about (or terminating in or otherwise comprising) the second extremum. In various embodiments, the tumor ROI may be a first number of base pairs on one or both sides of the first extremum, and the CH ROI may be a second number of base pairs on one or both sides of the second extremum. The first and second number of base pairs may be equal to each other or different from each other. The first and second numbers of base pairs may be selected based on the data. In an example, if an extremum is a very narrow peak, the corresponding ROI may also be narrow, and if another extremum is relatively much broader, the corresponding ROI may be wider as well.

In various embodiments, the gene mutation may be, for example, in a cancer-related gene including but not limited to: AKT1, ALK, APC, AR, ARAF, ARID1A, ARID2, ATM, B2M, BCL2, BCOR, BRAF, BRCA1, BRCA2, CARD11, CBFB, CCND1, CDH1, CDK4, CDKN2A, CIC, CREBBP, CTCF, CTNNB1, DICER1, DIS3, DNMT3A, EGFR, EIFIAX, EP300, ERBB2, ERBB3, ERCC2, ESR1, EZH2, FBXW7, FGFR1, FGFR2, FGFR3, FGFR4, FLT3, FOXA1, FOXL2, FOXO1, FUBP1, GATA3, GNA11, GNAQ, GNAS, H3F3A, HIST1H3B, HRAS, IDH1, IDH2, IKZF1, INPPL1, JAK1, KDM6A, KEAP1, KIT, KNSTRN, KRAS, MAP2K1, MAPK1, MAX, MED12, MET, MLH1, MSH2, MSH3, MSH6, MTOR, MYC, MYCN, MYD88, MYOD1, NF1, NFE2L2, NOTCH1, NRAS, NTRK1, NTRK2, NTRK3, NUP93, PAK7, PDGFRA, PIK3CA, PIK3CB, PIK3R1, PIK3R2, PMS2, POLE, PPP2R1A, PPP6C, PRKCI, PTCH1, PTEN, PTPN11, RAC1, RAF1, RB1, RET, RHOA, RIT1, ROS1, RRAS2, RXRA, SETD2, SF3B1, SMAD3, SMAD4, SMARCA4, SMARCB1, SOS1, SPOP, STAT3, STK11, STK19, TCF7L2, TERT, TGFBR1, TGFBR2, TP53, TP63, TSC1, TSC2, U2AF1, VHL, XPO1, or others.

In various embodiments, the predictive model may be trained on the tumor fragment size profile and the CH fragment size profile using supervised, semi-supervised, and/or unsupervised learning.

In another aspect, embodiments of the disclosure relate to a computing system for distinguishing tumor-derived mutations from clonal hematopoietic derived mutations in cell-free DNA (cfDNA). The computing system may comprise one or more processors configured to: acquire, from a sequencing device, sequence reads corresponding to cfDNA fragments in a sample of a test subject; detect, using the sequence reads corresponding to the cfDNA fragments, a gene mutation in the cfDNA; generate a size profile for a set of cfDNA fragments with the gene mutation, the size profile identifying how many cfDNA fragments are detected for each fragment length in a plurality of fragment lengths; classify, in the set of cfDNA fragments in the cfDNA sample, a first subset of cfDNA fragments as having a tumor origin and a second subset of cfDNA fragments as having a CH origin by feeding the size profile as an input to a predictive model that is configured to generate, for the gene mutation, a first set ranges of fragment lengths for fragments with the tumor origin and a second set of ranges of fragment lengths for fragments of the CH origin, wherein the first subset of cfDNA fragments have lengths falling in the first set of ranges and the second subset of cfDNA fragments have lengths falling in the second set of ranges; and generate a characterization of the mutation based on the classifying of cfDNA fragments. The characterization may be generated using a metric threshold.

In various embodiments, the one or more processors may be configured to train the predictive model by: acquiring, from the sequencing device, sequence reads corresponding to cfDNA fragments in samples of a plurality of subjects with known tumor mutations and/or known CH mutations; and generating, by the one or more processors, using the sequence reads from the sequencing device, a tumor fragment size profile and a CH fragment size profile.

In various embodiments, the one or more processors may further be configured to train the predictive model by analyzing the tumor fragment size profile and the CH fragment size profile to generate one or more tumor regions of interest (ROIs) and one or more CH ROIs. In various embodiments, analyzing the tumor and CH fragment size profiles may comprise applying smoothing operations to obtain trend lines. In various embodiments, a smoothing operation may be applied to the tumor fragment size profile to obtain a tumor trend line, and the smoothing operation may be applied to the CH fragment size profile to obtain a CH trend line. In various embodiments, a tumor ROI may be defined in the tumor trend line, and a CH ROI may be defined in the CH trend line. The tumor ROI may correspond with the first set of ranges of fragment lengths and the CH ROI may correspond with the second set of ranges of fragment lengths.

In various embodiments, the one or more processors may further be configured to determine a difference between the tumor fragment size profile and the CH fragment size profile. The smoothing operation may be applied to the difference to obtain a differential trend line. In various embodiments, the differential trend line may be obtained by determining a difference between the tumor trend line and the CH trend line.

In various embodiments, the one or more processors may further be configured to train the predictive model by generating, for each mutation, a metric based on the proportion of fragments in the tumor and CH ROIs. The metric may be a number of cfDNA fragments with lengths in the tumor ROIs divided by a total number of cfDNA fragments with lengths in both the tumor ROIs and the CH ROIs. The metric may alternatively be a number of cfDNA fragments with lengths in the CH ROIs divided by a total number of cfDNA fragments with lengths in the tumor ROIs and/or the CH ROIs

In various embodiments, the one or more processors may further be configured to select a metric threshold for use in classifying cfDNA fragments as having the tumor-derived mutation or the CH-derived mutation.

In another aspect, embodiments of the disclosure relate to a computer-implemented method to distinguish tumor-derived mutations from clonal hematopoietic derived mutations in cell-free DNA (cfDNA). The method may comprise: obtaining, by one or more processors, from a sequencing device, sequence reads corresponding to cfDNA fragments in samples of a plurality of subjects with known tumor mutations and/or known CH mutations; generating, by the one or more processors, using the sequence reads from the sequencing device, a tumor fragment size profile and a CH fragment size profile; computing, by the one or more processors, a difference between the tumor fragment size profile and the CH fragment size profile; applying, by the one or more processors, a smoothing operation to the difference to obtain a trend line with a first set of extrema and a second set of extrema; defining, by the one or more processors, one or more tumor ROIs centered about extrema in the first set of extrema, and one or more CH ROIs centered about extrema in the second set of extrema; generating, by the one or more processors, for each mutation, a metric based on the proportion of fragments in the tumor and CH ROIs; classifying, by the one or more processors, a mutation in cfDNA of a test subject as having either tumor origin or CH origin using the metric; and generating, by the one or more processors, a characterization of the mutation based on the classifying the mutation. The characterization may be generated using a metric threshold.

In various embodiments, the metric may be a number of cfDNA fragments with lengths in the tumor ROIs divided by a total number of cfDNA fragments with lengths in both the tumor ROIs and the CH ROIs. The metric may also be a number of cfDNA fragments with lengths in the CH ROIs divided by a total number of cfDNA fragments with lengths in both the tumor ROIs and the CH ROIs.

In various embodiments, the method may further comprise selecting a metric threshold for use in classifying the mutation in the cfDNA as having either tumor origin or CH origin.

In another aspect, other embodiments of the disclosure also relate to a computing system for distinguishing tumor-derived mutations from clonal hematopoietic derived mutations in cell-free DNA (cfDNA). The computing system may comprise one or more processors configured to: obtain, from a sequencing device, sequence reads corresponding to cfDNA fragments in samples of a plurality of subjects with known tumor mutations and/or known CH mutations; generate, using the sequence reads, a tumor fragment size profile and a CH fragment size profile; compute a difference between the tumor fragment size profile and the CH fragment size profile; apply a smoothing operation to the difference to obtain a trend line with a set of extrema comprising at least a first extremum and a second extremum; define a tumor ROI centered about (or terminating in or otherwise comprising) the first extremum, and a CH ROI centered about (or terminating in or otherwise comprising) the second extremum; generate, for each mutation, a metric based on the proportion of fragments in the tumor and CH ROIs; classify a mutation in cfDNA of a test subject as having either tumor origin or CH origin using a metric threshold; and generate a characterization of the tumor or mutation based on the classifying the mutation. It is noted that “first” and “second” does not necessarily correspond with an order in which the extrema appear in the trend line. The first extremum may thus appear before the second extremum, or the first extremum may appear after the second extremum, along a continuum of increasing fragment lengths. Accordingly, the size (length) of fragments in a tumor ROI (corresponding with the first extremum) may be smaller (shorter) than fragments in a CH ROI (corresponding with the second extremum), or the size of fragments in a tumor ROI may be larger than fragments in a CH ROI.

In various embodiments, the metric is a number of cfDNA fragments with lengths in the tumor ROI divided by a total number of cfDNA fragments with lengths in both the tumor ROI and the CH ROI.

In various embodiments, the one or more processors are further configured to select the metric threshold for use in classifying the mutation in the cfDNA as having either tumor origin or CH origin.

In another aspect, other embodiments of the disclosure also relate to a computer-implemented method to distinguish tumor-derived mutations from clonal hematopoietic (CH) derived mutations in cell-free DNA (cfDNA). The method may comprise obtaining, by one or more processors, from a sequencing device, sequence reads corresponding to cfDNA fragments in a cfDNA sample of a patient; detecting, by the one or more processors, using the sequence reads corresponding to the cfDNA fragments, a gene mutation in the cfDNA of the patient; generating, by the one or more processors, using the sequence reads corresponding to the cfDNA fragments of the patient, a size profile for the cfDNA fragments in the cfDNA sample; characterizing, by the one or more processors, the gene mutation as having either tumor origin or CH origin using a metric threshold generated by: obtaining sequence reads corresponding to cfDNA fragments in samples of a plurality of subjects with known tumor mutations and/or known CH mutations, generating, using the obtained sequence reads, a tumor fragment size profile and a CH fragment size profile; determining a difference between the tumor fragment size profile and the CH fragment size profile, applying a smoothing operation to the difference to obtain a trend line with a set of one or more maximums and one or more minimums, defining a tumor ROI centered about (or terminating in or otherwise comprising) a first maximum or minimum in the set, and a CH ROI centered about (or terminating in or otherwise comprising) a second maximum or minimum in the set, and generating, for the mutation, the metric threshold based on a proportion of fragments in the tumor and CH ROIs; classifying, by the one or more processors, the cfDNA fragments of the patient as having either tumor origin or CH origin using the tumor ROIs and CH ROIs; generating, by the one or more processors, a metric for the cfDNA fragments of the patient based on the classifying the cfDNA fragments of the patient; and generating, by the one or more processors, a characterization of the mutation using the metric and the metric threshold.

In various embodiments, the metric is a number of cfDNA fragments with lengths in one of the tumor ROI or the CH ROI, divided by a total number of cfDNA fragments with lengths in both the tumor ROI and the CH ROI.

In another aspect, various embodiments of the disclosure may relate to a method comprising: (a) extracting cell-free DNA (cfDNA) comprising tumor-origin cfDNA fragments and CH-origin cfDNA fragments from substantially cell-free samples of blood plasma and/or blood serum of a plurality of subjects; (b) producing one or more tumor regions of interest (ROIs) and one or more CH ROIs for the cfDNA fragments of (a) by: (i) generating a tumor fragment size profile and a CH fragment size profile; (ii) applying a smoothing operation to a difference between the tumor fragment size profile and the CH fragment size profile to obtain a trend line with a set of extrema comprising one or more maximums and one or more minimums; and (iii) defining the tumor and CH ROIs as sets of ranges of cfDNA fragment sizes based on the maximums and minimums; and (c) extracting and analyzing cfDNA fragments in a sample of a patient using the tumor and CH ROIs.

In various embodiments, the method may further comprise generating a metric threshold using the samples of the plurality of subjects, determining a metric for the sample of the patient, and characterizing the cfDNA fragments in the sample of the patient by comparing the metric with the metric threshold.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIG. 1A is a block diagram depicting an embodiment of a network environment comprising a client device in communication with server device.

FIG. 1B is a block diagram depicting a cloud computing environment comprising client device in communication with cloud service providers.

FIGS. 1C and 1D are block diagrams depicting embodiments of computing devices useful in connection with the methods and systems described herein.

FIGS. 2A-2C depict fragment size analysis of reads bearing mutations derived from tumor and clonal hematopoiesis in plasma cfDNA, according to example embodiments. FIG. 2A shows relative enrichment between tumor (positive values) and CH fragments (negative values), obtained by subtracting the normalized CH size profile from the normalized tumor profile. The black line is a LOESS fit. Shaded areas denote the selected regions of interest (tumor and CH). FIG. 2B provides distribution of the proportion of fragments in tumor-enriched regions by class. FIG. 2C provides classification performance based on the statistic shown in FIG. 2B, with an Area Under the Curve (AUC) of 0.8089.

FIG. 3 illustrates a flow diagram of an example mutation classification process according to potential embodiments.

FIG. 4 depicts the performance of a classifier assessed via the area under the receiver operating characteristic (ROC) curve (AUC) for 1000 randomized datasets. The dataset (corresponding to the cohort of FIGS. 2A-2C) was randomly split into a training set and a test set 1000 times, maintaining the proportions of the two classes (CH and tumor) in both sets. ROIs were then automatically discovered in the training set, and classification based on those ROIs was performed on the test set. Classification is thus performed on data that was not used to define the ROIs.

FIG. 5 depicts a representative predictive model for classifying and characterizing mutations according to potential embodiments.

FIG. 6 illustrates a flow diagram of an example process for training a predictive model according to potential embodiments.

FIG. 7 depicts a system that includes a computing device and a sample processing system according to potential embodiments.

### DETAILED DESCRIPTION

For purposes of reading the description of the various embodiments below, the following descriptions of the sections of the specification and their respective contents may be helpful:

Section A describes a network environment and computing environment which may be useful for practicing embodiments described herein.

Section B describes methods for identifying mutations in cell-free DNA.

Section C describes embodiments of systems and methods of the present technology for distinguishing mutations of tumor origin from CH-mutations in cell-free DNA.

The disclosed approach enhances accuracy (sensitivity and specificity) in detection of pathogenic mutations (e.g., tumor-derived mutations) in cfDNA by reducing false positive tumor calls due to mutations derived from the hematopoietic system (e.g., mutations due to clonal hematopoiesis). Exemplary embodiments are directed to methods for discriminating between mutations derived from tumors and mutations derived of CH origin by analyzing the fragment sizes of cfDNA. Such CH mutations are often found in cancer driver genes that have therapeutic implications, and if mistaken as being tumor-derived, they may lead to wrong or otherwise non-ideal clinical decisions. It has been shown in the literature that, in plasma, cfDNA of tumor origin tends to be on average shorter than DNA derived from white blood cells. That is, literature data have shown that the lengths of cfDNA fragments bearing a tumor-specific mutation may be shorter than cfDNA fragments that do not bear a tumor mutation (i.e., wild type DNA), which are believed to be derived from white blood cells. A GRAIL, Inc. study (Hubbell et al., “Cell-free DNA (cfDNA) Fragment Length Patterns of Tumor- and Blood-derived Variants in Participants With and Without Cancer”, Cancer Research 79(13): Abstract 3372 (2019)) defined fragment size distributions for two groups of mutations: WM (WBC-matched, namely CH mutations), and TBM (tumor biopsy-matched, namely tumor mutations). Instead of highlighting ROIs, however, the Grail study considered the entire fragment size profile, whether informative or not, and for each mutation calculated the probability that the fragments supporting the mutation were generated from each size distribution. Based on these probabilities, it was determined whether it was more likely that the mutation came from one group or the other. The GRAIL study concluded that, “because most fragment length distributions from varied sources overlapped, fragment length alone did not strongly distinguish tumor-derived from WBC-derived variants.”

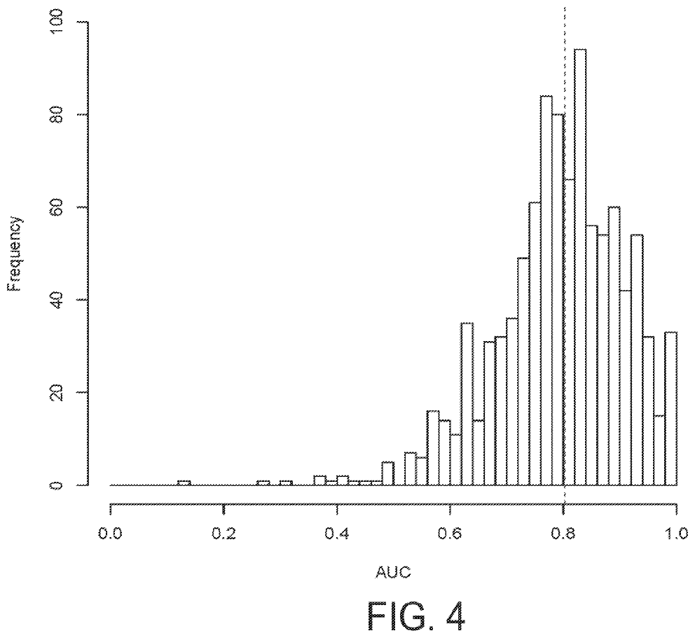

The robustness of the disclosed approach is demonstrated by randomly splitting the dataset into a training set and a test set, and maintaining the proportions of the two classes (CH and tumor) in both sets in this example analysis. ROIs are automatically discovered in the training set, and classification based on those ROIs are performed on the test set. Classification is performed on data that were not used to define the ROIs. The performance of the classifier may be assessed via the area under the receiver operating characteristic (ROC) curve (AUC). Because some random splits of the dataset may be more favorable than others, the process was repeated 1000 times. FIG. 4 provides a distribution of the resulting AUCs. Despite the smaller training sets, and with an automated ROI discovery, average performance remained at about 0.80.

A. Computing and Network Environment

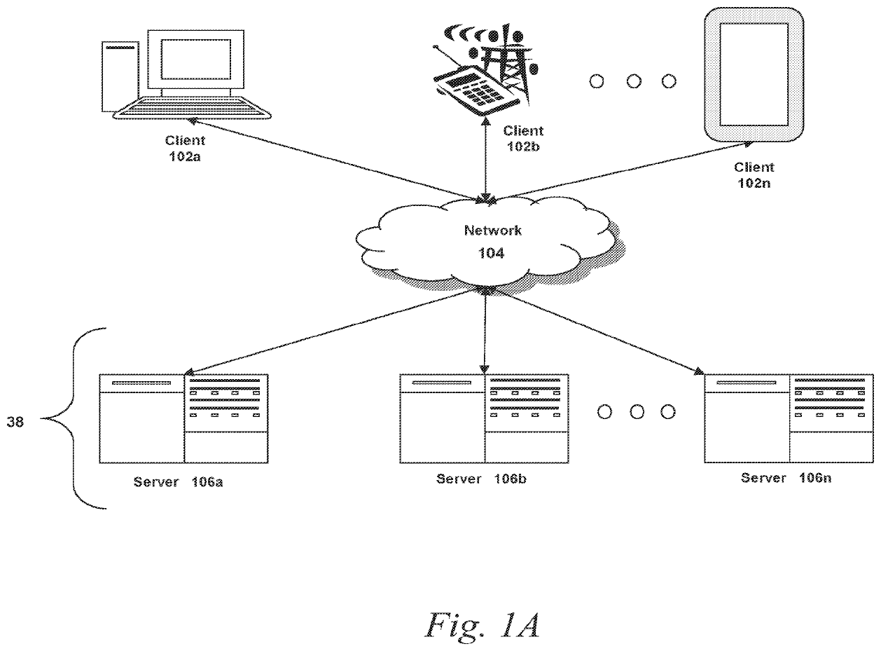

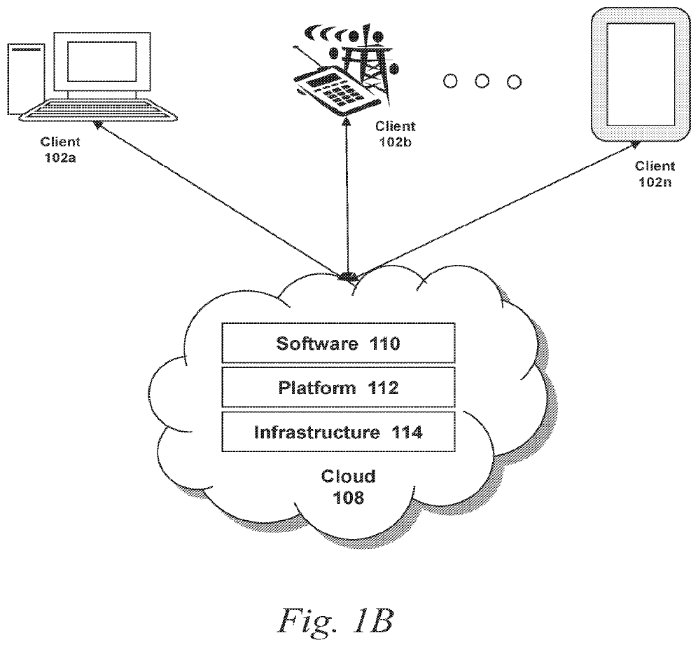

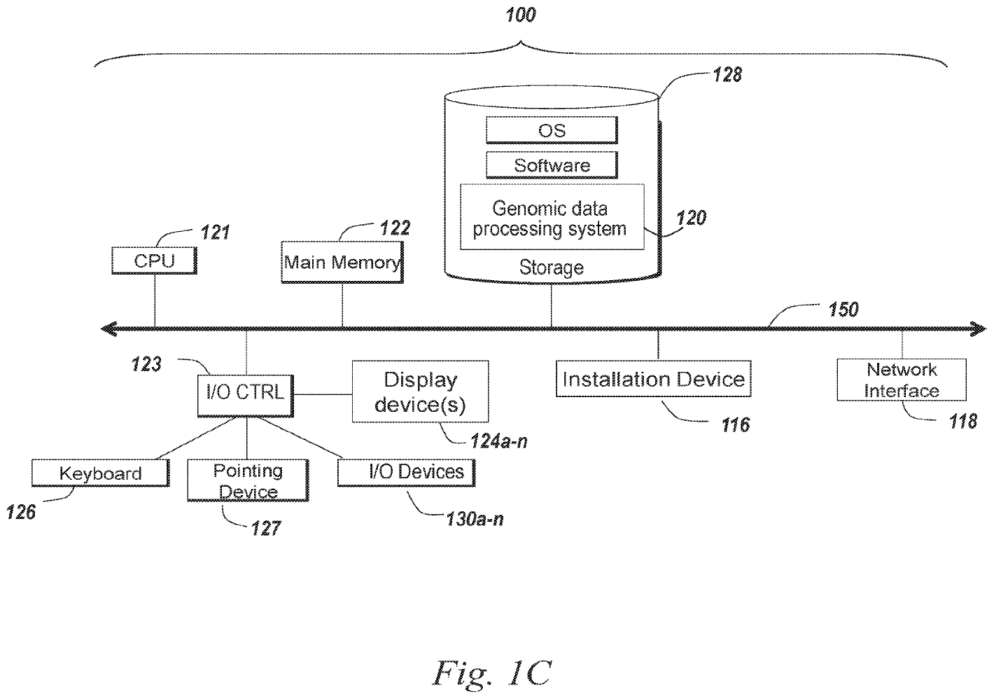

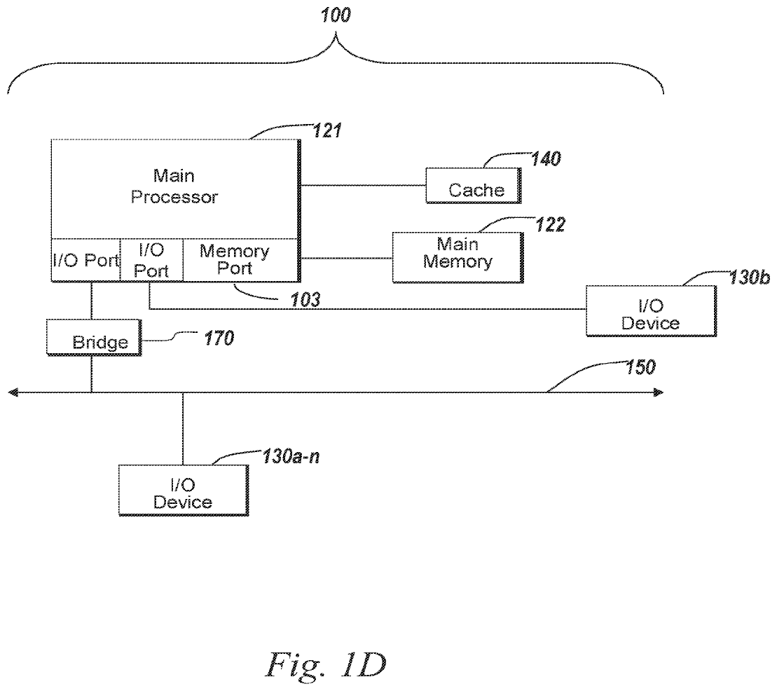

Prior to discussing specific embodiments of the present solution, it may be helpful to describe aspects of the operating environment as well as associated system components (e.g., hardware elements) in connection with the methods and systems described herein. Referring to FIG. 1A, an embodiment of a network environment is depicted. In brief overview, the network environment includes one or more clients 102a-102n (also generally referred to as local machine(s) 102, client(s) 102, client node(s) 102, client machine(s) 102, client computer(s) 102, client device(s) 102, endpoint(s) 102, or endpoint node(s) 102) in communication with one or more servers 106a-106n (also generally referred to as server(s) 106, node 106, or remote machine(s) 106) via one or more networks 104. In some embodiments, a client 102 has the capacity to function as both a client node seeking access to resources provided by a server and as a server providing access to hosted resources for other clients 102a-102n.

Although FIG. 1A shows a network 104 between the clients 102 and the servers 106, the clients 102 and the servers 106 may be on the same network 104. In some embodiments, there are multiple networks 104 between the clients 102 and the servers 106. In one of these embodiments, a network 104′ (not shown) may be a private network and a network 104 may be a public network. In another of these embodiments, a network 104 may be a private network and a network 104′ a public network. In still another of these embodiments, networks 104 and 104′ may both be private networks.

The network 104 may be connected via wired or wireless links. Wired links may include Digital Subscriber Line (DSL), coaxial cable lines, or optical fiber lines. The wireless links may include BLUETOOTH, Wi-Fi, Worldwide Interoperability for Microwave Access (WiMAX), an infrared channel or satellite band. The wireless links may also include any cellular network standards used to communicate among mobile devices, including standards that qualify as 1G, 2G, 3G, or 4G. The network standards may qualify as one or more generation of mobile telecommunication standards by fulfilling a specification or standards such as the specifications maintained by International Telecommunication Union. The 3G standards, for example, may correspond to the International Mobile Telecommunications-2000 (IMT-2000) specification, and the 4G standards may correspond to the International Mobile Telecommunications Advanced (IMT-Advanced) specification. Examples of cellular network standards include AMPS, GSM, GPRS, UMTS, LTE, LTE Advanced, Mobile WiMAX, and WiMAX-Advanced. Cellular network standards may use various channel access methods e.g. FDMA, TDMA, CDMA, or SDMA. In some embodiments, different types of data may be transmitted via different links and standards. In other embodiments, the same types of data may be transmitted via different links and standards.

The network 104 may be any type and/or form of network. The geographical scope of the network 104 may vary widely and the network 104 can be a body area network (BAN), a personal area network (PAN), a local-area network (LAN), e.g. Intranet, a metropolitan area network (MAN), a wide area network (WAN), or the Internet. The topology of the network 104 may be of any form and may include, e.g., any of the following: point-to-point, bus, star, ring, mesh, or tree. The network 104 may be an overlay network which is virtual and sits on top of one or more layers of other networks 104′. The network 104 may be of any such network topology as known to those ordinarily skilled in the art capable of supporting the operations described herein. The network 104 may utilize different techniques and layers or stacks of protocols, including, e.g., the Ethernet protocol, the internet protocol suite (TCP/IP), the ATM (Asynchronous Transfer Mode) technique, the SONET (Synchronous Optical Networking) protocol, or the SDH (Synchronous Digital Hierarchy) protocol. The TCP/IP internet protocol suite may include application layer, transport layer, internet layer (including, e.g., IPv6), or the link layer. The network 104 may be a type of a broadcast network, a telecommunications network, a data communication network, or a computer network.

In some embodiments, the system may include multiple, logically-grouped servers 106. In one of these embodiments, the logical group of servers may be referred to as a server farm 38 or a machine farm 38. In another of these embodiments, the servers 106 may be geographically dispersed. In other embodiments, a machine farm 38 may be administered as a single entity. In still other embodiments, the machine farm 38 includes a plurality of machine farms 38. The servers 106 within each machine farm 38 can be heterogeneous—one or more of the servers 106 or machines 106 can operate according to one type of operating system platform (e.g., WINDOWS NT, manufactured by Microsoft Corp. of Redmond, Wash.), while one or more of the other servers 106 can operate on according to another type of operating system platform (e.g., Unix, Linux, or Mac OS X).

In one embodiment, servers 106 in the machine farm 38 may be stored in high-density rack systems, along with associated storage systems, and located in an enterprise data center. In this embodiment, consolidating the servers 106 in this way may improve system manageability, data security, the physical security of the system, and system performance by locating servers 106 and high performance storage systems on localized high performance networks. Centralizing the servers 106 and storage systems and coupling them with advanced system management tools allows more efficient use of server resources.

The servers 106 of each machine farm 38 do not need to be physically proximate to another server 106 in the same machine farm 38. Thus, the group of servers 106 logically grouped as a machine farm 38 may be interconnected using a wide-area network (WAN) connection or a metropolitan-area network (MAN) connection. For example, a machine farm 38 may include servers 106 physically located in different continents or different regions of a continent, country, state, city, campus, or room. Data transmission speeds between servers 106 in the machine farm 38 can be increased if the servers 106 are connected using a local-area network (LAN) connection or some form of direct connection. Additionally, a heterogeneous machine farm 38 may include one or more servers 106 operating according to a type of operating system, while one or more other servers 106 execute one or more types of hypervisors rather than operating systems. In these embodiments, hypervisors may be used to emulate virtual hardware, partition physical hardware, virtualize physical hardware, and execute virtual machines that provide access to computing environments, allowing multiple operating systems to run concurrently on a host computer. Native hypervisors may run directly on the host computer. Hypervisors may include VMware ESX/ESXi, manufactured by VMWare, Inc., of Palo Alto, Calif.; the Xen hypervisor, an open source product whose development is overseen by Citrix Systems, Inc.; the HYPER-V hypervisors provided by Microsoft or others. Hosted hypervisors may run within an operating system on a second software level. Examples of hosted hypervisors may include VMware Workstation and VIRTUALBOX.

Management of the machine farm 38 may be de-centralized. For example, one or more servers 106 may comprise components, subsystems and modules to support one or more management services for the machine farm 38. In one of these embodiments, one or more servers 106 provide functionality for management of dynamic data, including techniques for handling failover, data replication, and increasing the robustness of the machine farm 38. Each server 106 may communicate with a persistent store and, in some embodiments, with a dynamic store.

Server 106 may be a file server, application server, web server, proxy server, appliance, network appliance, gateway, gateway server, virtualization server, deployment server, SSL VPN server, or firewall. In one embodiment, the server 106 may be referred to as a remote machine or a node. In another embodiment, a plurality of nodes 290 may be in the path between any two communicating servers.

Referring to FIG. 1B, a cloud computing environment is depicted. A cloud computing environment may provide client 102 with one or more resources provided by a network environment. The cloud computing environment may include one or more clients 102a-102n, in communication with the cloud 108 over one or more networks 104. Clients 102 may include, e.g., thick clients, thin clients, and zero clients. A thick client may provide at least some functionality even when disconnected from the cloud 108 or servers 106. A thin client or a zero client may depend on the connection to the cloud 108 or server 106 to provide functionality. A zero client may depend on the cloud 108 or other networks 104 or servers 106 to retrieve operating system data for the client device. The cloud 108 may include back end platforms, e.g., servers 106, storage, server farms or data centers.

The cloud 108 may be public, private, or hybrid. Public clouds may include public servers 106 that are maintained by third parties to the clients 102 or the owners of the clients. The servers 106 may be located off-site in remote geographical locations as disclosed above or otherwise. Public clouds may be connected to the servers 106 over a public network. Private clouds may include private servers 106 that are physically maintained by clients 102 or owners of clients. Private clouds may be connected to the servers 106 over a private network 104. Hybrid clouds 108 may include both the private and public networks 104 and servers 106.

The cloud 108 may also include a cloud based delivery, e.g. Software as a Service (SaaS) 110, Platform as a Service (PaaS) 112, and Infrastructure as a Service (IaaS) 114. IaaS may refer to a user renting the use of infrastructure resources that are needed during a specified time period. IaaS providers may offer storage, networking, servers or virtualization resources from large pools, allowing the users to quickly scale up by accessing more resources as needed. Examples of IaaS can include infrastructure and services (e.g., EG-32) provided by OVH HOSTING of Montreal, Quebec, Canada, AMAZON WEB SERVICES provided by Amazon.com, Inc., of Seattle, Wash., RACKSPACE CLOUD provided by Rackspace US, Inc., of San Antonio, Tex., Google Compute Engine provided by Google Inc. of Mountain View, Calif., or RIGHTSCALE provided by RightScale, Inc., of Santa Barbara, Calif. PaaS providers may offer functionality provided by IaaS, including, e.g., storage, networking, servers or virtualization, as well as additional resources such as, e.g., the operating system, middleware, or runtime resources. Examples of PaaS include WINDOWS AZURE provided by Microsoft Corporation of Redmond, Wash., Google App Engine provided by Google Inc., and HEROKU provided by Heroku, Inc. of San Francisco, Calif. SaaS providers may offer the resources that PaaS provides, including storage, networking, servers, virtualization, operating system, middleware, or runtime resources. In some embodiments, SaaS providers may offer additional resources including, e.g., data and application resources. Examples of SaaS include GOOGLE APPS provided by Google Inc., SALESFORCE provided by Salesforce.com Inc. of San Francisco, Calif., or OFFICE 365 provided by Microsoft Corporation. Examples of SaaS may also include data storage providers, e.g. DROPBOX provided by Dropbox, Inc. of San Francisco, Calif., Microsoft SKYDRIVE provided by Microsoft Corporation, Google Drive provided by Google Inc., or Apple ICLOUD provided by Apple Inc. of Cupertino, Calif.

Clients 102 may access IaaS resources with one or more IaaS standards, including, e.g., Amazon Elastic Compute Cloud (EC2), Open Cloud Computing Interface (OCCI), Cloud Infrastructure Management Interface (CIMI), or OpenStack standards. Some IaaS standards may allow clients access to resources over HTTP, and may use Representational State Transfer (REST) protocol or Simple Object Access Protocol (SOAP). Clients 102 may access PaaS resources with different PaaS interfaces. Some PaaS interfaces use HTTP packages, standard Java APIs, JavaMail API, Java Data Objects (JDO), Java Persistence API (JPA), Python APIs, web integration APIs for different programming languages including, e.g., Rack for Ruby, WSGI for Python, or PSGI for Perl, or other APIs that may be built on REST, HTTP, XML, or other protocols. Clients 102 may access SaaS resources through the use of web-based user interfaces, provided by a web browser (e.g. GOOGLE CHROME, Microsoft INTERNET EXPLORER, or Mozilla Firefox provided by Mozilla Foundation of Mountain View, Calif.). Clients 102 may also access SaaS resources through smartphone or tablet applications, including, e.g., Salesforce Sales Cloud, or Google Drive app. Clients 102 may also access SaaS resources through the client operating system, including, e.g., Windows file system for DROPBOX.

In some embodiments, access to IaaS, PaaS, or SaaS resources may be authenticated. For example, a server or authentication server may authenticate a user via security certificates, HTTPS, or API keys. API keys may include various encryption standards such as, e.g., Advanced Encryption Standard (AES). Data resources may be sent over Transport Layer Security (TLS) or Secure Sockets Layer (SSL).

The client 102 and server 106 may be deployed as and/or executed on any type and form of computing device, e.g. a computer, network device or appliance capable of communicating on any type and form of network and performing the operations described herein. FIGS. 1C and 1D depict block diagrams of a computing device 100 useful for practicing an embodiment of the client 102 or a server 106. As shown in FIGS. 1C and 1D, each computing device 100 includes a central processing unit 121, and a main memory unit 122. As shown in FIG. 1C, a computing device 100 may include a storage device 128, an installation device 116, a network interface 118, an I/O controller 123, display devices 124a-124n, a keyboard 126 and a pointing device 127, e.g. a mouse. The storage device 128 may include, without limitation, an operating system, software, and a software of a genomic data processing system 120. As shown in FIG. 1D, each computing device 100 may also include additional optional elements, e.g. a memory port 103, a bridge 170, one or more input/output devices 130a-130n (generally referred to using reference numeral 130), and a cache memory 140 in communication with the central processing unit 121.

The central processing unit 121 is any logic circuitry that responds to and processes instructions fetched from the main memory unit 122. In many embodiments, the central processing unit 121 is provided by a microprocessor unit, e.g.: those manufactured by Intel Corporation of Mountain View, Calif.; those manufactured by Motorola Corporation of Schaumburg, Ill.; the ARM processor and TEGRA system on a chip (SoC) manufactured by Nvidia of Santa Clara, Calif.; the POWER7 processor, those manufactured by International Business Machines of White Plains, N.Y.; or those manufactured by Advanced Micro Devices of Sunnyvale, Calif. The computing device 100 may be based on any of these processors, or any other processor capable of operating as described herein. The central processing unit 121 may utilize instruction level parallelism, thread level parallelism, different levels of cache, and multi-core processors. A multi-core processor may include two or more processing units on a single computing component. Examples of multi-core processors include the AMD PHENOM IIX2, INTEL CORE i5 and INTEL CORE i7.

Main memory unit 122 may include one or more memory chips capable of storing data and allowing any storage location to be directly accessed by the microprocessor 121. Main memory unit 122 may be volatile and faster than storage 128 memory. Main memory units 122 may be Dynamic random access memory (DRAM) or any variants, including static random access memory (SRAM), Burst SRAM or SynchBurst SRAM (BSRAM), Fast Page Mode DRAM (FPM DRAM), Enhanced DRAM (EDRAM), Extended Data Output RAM (EDO RAM), Extended Data Output DRAM (EDO DRAM), Burst Extended Data Output DRAM (BEDO DRAM), Single Data Rate Synchronous DRAM (SDR SDRAM), Double Data Rate SDRAM (DDR SDRAM), Direct Rambus DRAM (DRDRAM), or Extreme Data Rate DRAM (XDR DRAM). In some embodiments, the main memory 122 or the storage 128 may be non-volatile; e.g., non-volatile read access memory (NVRAM), flash memory non-volatile static RAM (nvSRAM), Ferroelectric RAM (FeRAM), Magnetoresistive RAM (MRAM), Phase-change memory (PRAM), conductive-bridging RAM (CBRAM), Silicon-Oxide-Nitride-Oxide-Silicon (SONOS), Resistive RAM (RRAM), Racetrack, Nano-RAM (NRAM), or Millipede memory. The main memory 122 may be based on any of the above described memory chips, or any other available memory chips capable of operating as described herein. In the embodiment shown in FIG. 1C, the processor 121 communicates with main memory 122 via a system bus 150 (described in more detail below). FIG. 1D depicts an embodiment of a computing device 100 in which the processor communicates directly with main memory 122 via a memory port 103. For example, in FIG. 1D the main memory 122 may be DRDRAM.

FIG. 1D depicts an embodiment in which the main processor 121 communicates directly with cache memory 140 via a secondary bus, sometimes referred to as a backside bus. In other embodiments, the main processor 121 communicates with cache memory 140 using the system bus 150. Cache memory 140 typically has a faster response time than main memory 122 and is typically provided by SRAM, BSRAM, or EDRAM. In the embodiment shown in FIG. 1D, the processor 121 communicates with various I/O devices 130 via a local system bus 150. Various buses may be used to connect the central processing unit 121 to any of the I/O devices 130, including a PCI bus, a PCI-X bus, or a PCI-Express bus, or a NuBus. For embodiments in which the I/O device is a video display 124, the processor 121 may use an Advanced Graphics Port (AGP) to communicate with the display 124 or the I/O controller 123 for the display 124. FIG. 1D depicts an embodiment of a computer 100 in which the main processor 121 communicates directly with I/O device 130b or other processors 121′ via HYPERTRANSPORT, RAPIDIO, or INFINIBAND communications technology. FIG. 1D also depicts an embodiment in which local busses and direct communication are mixed: the processor 121 communicates with I/O device 130a using a local interconnect bus while communicating with I/O device 130b directly.

A wide variety of I/O devices 130a-130n may be present in the computing device 100. Input devices may include keyboards, mice, trackpads, trackballs, touchpads, touch mice, multi-touch touchpads and touch mice, microphones, multi-array microphones, drawing tablets, cameras, single-lens reflex camera (SLR), digital SLR (DSLR), CMOS sensors, accelerometers, infrared optical sensors, pressure sensors, magnetometer sensors, angular rate sensors, depth sensors, proximity sensors, ambient light sensors, gyroscopic sensors, or other sensors. Output devices may include video displays, graphical displays, speakers, headphones, inkjet printers, laser printers, and 3D printers.

Devices 130a-130n may include a combination of multiple input or output devices, including, e.g., Microsoft KINECT, Nintendo Wiimote for the WII, Nintendo WII U GAMEPAD, or Apple IPHONE. Some devices 130a-130n allow gesture recognition inputs through combining some of the inputs and outputs. Some devices 130a-130n provides for facial recognition which may be utilized as an input for different purposes including authentication and other commands. Some devices 130a-130n provides for voice recognition and inputs, including, e.g., Microsoft KINECT, SIRI for IPHONE by Apple, Google Now or Google Voice Search.

Additional devices 130a-130n have both input and output capabilities, including, e.g., haptic feedback devices, touchscreen displays, or multi-touch displays. Touchscreen, multi-touch displays, touchpads, touch mice, or other touch sensing devices may use different technologies to sense touch, including, e.g., capacitive, surface capacitive, projected capacitive touch (PCT), in-cell capacitive, resistive, infrared, waveguide, dispersive signal touch (DST), in-cell optical, surface acoustic wave (SAW), bending wave touch (BWT), or force-based sensing technologies. Some multi-touch devices may allow two or more contact points with the surface, allowing advanced functionality including, e.g., pinch, spread, rotate, scroll, or other gestures. Some touchscreen devices, including, e.g., Microsoft PIXELSENSE or Multi-Touch Collaboration Wall, may have larger surfaces, such as on a table-top or on a wall, and may also interact with other electronic devices. Some I/O devices 130a-130n, display devices 124a-124n or group of devices may be augment reality devices. The I/O devices may be controlled by an I/O controller 123 as shown in FIG. 1C. The I/O controller may control one or more I/O devices, such as, e.g., a keyboard 126 and a pointing device 127, e.g., a mouse or optical pen. Furthermore, an I/O device may also provide storage and/or an installation medium 116 for the computing device 100. In still other embodiments, the computing device 100 may provide USB connections (not shown) to receive handheld USB storage devices. In further embodiments, an I/O device 130 may be a bridge between the system bus 150 and an external communication bus, e.g. a USB bus, a SCSI bus, a FireWire bus, an Ethernet bus, a Gigabit Ethernet bus, a Fibre Channel bus, or a Thunderbolt bus.

In some embodiments, display devices 124a-124n may be connected to I/O controller 123. Display devices may include, e.g., liquid crystal displays (LCD), thin film transistor LCD (TFT-LCD), blue phase LCD, electronic papers (e-ink) displays, flexile displays, light emitting diode displays (LED), digital light processing (DLP) displays, liquid crystal on silicon (LCOS) displays, organic light-emitting diode (OLED) displays, active-matrix organic light-emitting diode (AMOLED) displays, liquid crystal laser displays, time-multiplexed optical shutter (TMOS) displays, or 3D displays. Examples of 3D displays may use, e.g. stereoscopy, polarization filters, active shutters, or autostereoscopy. Display devices 124a-124n may also be a head-mounted display (TIMD). In some embodiments, display devices 124a-124n or the corresponding I/O controllers 123 may be controlled through or have hardware support for OPENGL or DIRECTX API or other graphics libraries.

In some embodiments, the computing device 100 may include or connect to multiple display devices 124a-124n, which each may be of the same or different type and/or form. As such, any of the I/O devices 130a-130n and/or the I/O controller 123 may include any type and/or form of suitable hardware, software, or combination of hardware and software to support, enable or provide for the connection and use of multiple display devices 124a-124n by the computing device 100. For example, the computing device 100 may include any type and/or form of video adapter, video card, driver, and/or library to interface, communicate, connect or otherwise use the display devices 124a-124n. In one embodiment, a video adapter may include multiple connectors to interface to multiple display devices 124a-124n. In other embodiments, the computing device 100 may include multiple video adapters, with each video adapter connected to one or more of the display devices 124a-124n. In some embodiments, any portion of the operating system of the computing device 100 may be configured for using multiple displays 124a-124n. In other embodiments, one or more of the display devices 124a-124n may be provided by one or more other computing devices 100a or 100b connected to the computing device 100, via the network 104. In some embodiments software may be designed and constructed to use another computer's display device as a second display device 124a for the computing device 100. For example, in one embodiment, an Apple iPad may connect to a computing device 100 and use the display of the device 100 as an additional display screen that may be used as an extended desktop. One ordinarily skilled in the art will recognize and appreciate the various ways and embodiments that a computing device 100 may be configured to have multiple display devices 124a-124n.

Referring again to FIG. 1C, the computing device 100 may comprise a storage device 128 (e.g. one or more hard disk drives or redundant arrays of independent disks) for storing an operating system or other related software, and for storing application software programs such as any program related to the software for the genomic data processing system 120. Examples of storage device 128 include, e.g., hard disk drive (HDD); optical drive including CD drive, DVD drive, or BLU-RAY drive; solid-state drive (SSD); USB flash drive; or any other device suitable for storing data. Some storage devices may include multiple volatile and non-volatile memories, including, e.g., solid state hybrid drives that combine hard disks with solid state cache. Some storage device 128 may be non-volatile, mutable, or read-only. Some storage device 128 may be internal and connect to the computing device 100 via a bus 150. Some storage devices 128 may be external and connect to the computing device 100 via an I/O device 130 that provides an external bus. Some storage device 128 may connect to the computing device 100 via the network interface 118 over a network 104, including, e.g., the Remote Disk for MACBOOK AIR by Apple. Some client devices 100 may not require a non-volatile storage device 128 and may be thin clients or zero clients 102. Some storage device 128 may also be used as an installation device 116, and may be suitable for installing software and programs. Additionally, the operating system and the software can be run from a bootable medium, for example, a bootable CD, e.g. KNOPPIX, a bootable CD for GNU/Linux that is available as a GNU/Linux distribution from knoppix.net.

Client device 100 may also install software or application from an application distribution platform. Examples of application distribution platforms include the App Store for iOS provided by Apple, Inc., the Mac App Store provided by Apple, Inc., GOOGLE PLAY for Android OS provided by Google Inc., Chrome Webstore for CHROME OS provided by Google Inc., and Amazon Appstore for Android OS and KINDLE FIRE provided by Amazon.com, Inc. An application distribution platform may facilitate installation of software on a client device 102. An application distribution platform may include a repository of applications on a server 106 or a cloud 108, which the clients 102a-102n may access over a network 104. An application distribution platform may include application developed and provided by various developers. A user of a client device 102 may select, purchase and/or download an application via the application distribution platform.

Furthermore, the computing device 100 may include a network interface 118 to interface to the network 104 through a variety of connections including, but not limited to, standard telephone lines LAN or WAN links (e.g., 802.11, T1, T3, Gigabit Ethernet, Infiniband), broadband connections (e.g., ISDN, Frame Relay, ATM, Gigabit Ethernet, Ethernet-over-SONET, ADSL, VDSL, BPON, GPON, fiber optical including FiOS), wireless connections, or some combination of any or all of the above. Connections can be established using a variety of communication protocols (e.g., TCP/IP, Ethernet, ARCNET, SONET, SDH, Fiber Distributed Data Interface (FDDI), IEEE 802.11a/b/g/n/ac CDMA, GSM, WiMax and direct asynchronous connections). In one embodiment, the computing device 100 communicates with other computing devices 100′ via any type and/or form of gateway or tunneling protocol e.g. Secure Socket Layer (SSL) or Transport Layer Security (TLS), or the Citrix Gateway Protocol manufactured by Citrix Systems, Inc. of Ft. Lauderdale, Fla. The network interface 118 may comprise a built-in network adapter, network interface card, PCMCIA network card, EXPRESSCARD network card, card bus network adapter, wireless network adapter, USB network adapter, modem or any other device suitable for interfacing the computing device 100 to any type of network capable of communication and performing the operations described herein.

A computing device 100 of the sort depicted in FIGS. 1B and 1C may operate under the control of an operating system, which controls scheduling of tasks and access to system resources. The computing device 100 can be running any operating system such as any of the versions of the MICROSOFT WINDOWS operating systems, the different releases of the Unix and Linux operating systems, any version of the MAC OS for Macintosh computers, any embedded operating system, any real-time operating system, any open source operating system, any proprietary operating system, any operating systems for mobile computing devices, or any other operating system capable of running on the computing device and performing the operations described herein. Typical operating systems include, but are not limited to: WINDOWS 2000, WINDOWS Server 2022, WINDOWS CE, WINDOWS Phone, WINDOWS XP, WINDOWS VISTA, and WINDOWS 7, WINDOWS RT, and WINDOWS 8 all of which are manufactured by Microsoft Corporation of Redmond, Wash.; MAC OS and iOS, manufactured by Apple, Inc. of Cupertino, Calif.; and Linux, a freely-available operating system, e.g. Linux Mint distribution (“distro”) or Ubuntu, distributed by Canonical Ltd. of London, United Kingdom; or Unix or other Unix-like derivative operating systems; and Android, designed by Google, of Mountain View, Calif., among others. Some operating systems, including, e.g., the CHROME OS by Google, may be used on zero clients or thin clients, including, e.g., CHROMEBOOKS.

The computer system 100 can be any workstation, telephone, desktop computer, laptop or notebook computer, netbook, ULTRABOOK, tablet, server, handheld computer, mobile telephone, smartphone or other portable telecommunications device, media playing device, a gaming system, mobile computing device, or any other type and/or form of computing, telecommunications or media device that is capable of communication. The computer system 100 has sufficient processor power and memory capacity to perform the operations described herein. The computer system 100 can be of any suitable size, such as a standard desktop computer or a Raspberry Pi 4 manufactured by Raspberry Pi Foundation, of Cambridge, United Kingdom. In some embodiments, the computing device 100 may have different processors, operating systems, and input devices consistent with the device. The Samsung GALAXY smartphones, e.g., operate under the control of Android operating system developed by Google, Inc. GALAXY smartphones receive input via a touch interface.

In some embodiments, the computing device 100 is a gaming system. For example, the computer system 100 may comprise a PLAYSTATION 3, or PERSONAL PLAYSTATION PORTABLE (PSP), or a PLAYSTATION VITA device manufactured by the Sony Corporation of Tokyo, Japan, a NINTENDO DS, NINTENDO 3DS, NINTENDO WII, or a NINTENDO WII U device manufactured by Nintendo Co., Ltd., of Kyoto, Japan, an XBOX 360 device manufactured by the Microsoft Corporation of Redmond, Wash.

In some embodiments, the computing device 100 is a digital audio player such as the Apple IPOD, IPOD Touch, and IPOD NANO lines of devices, manufactured by Apple Computer of Cupertino, Calif. Some digital audio players may have other functionality, including, e.g., a gaming system or any functionality made available by an application from a digital application distribution platform. For example, the IPOD Touch may access the Apple App Store. In some embodiments, the computing device 100 is a portable media player or digital audio player supporting file formats including, but not limited to, MP3, WAV, M4A/AAC, WMA Protected AAC, AIFF, Audible audiobook, Apple Lossless audio file formats and .mov, .m4v, and .mp4 MPEG-4 (H.264/MPEG-4 AVC) video file formats.

In some embodiments, the computing device 100 is a tablet e.g. the IPAD line of devices by Apple; GALAXY TAB family of devices by Samsung; or KINDLE FIRE, by Amazon.com, Inc. of Seattle, Wash. In other embodiments, the computing device 100 is an eBook reader, e.g. the KINDLE family of devices by Amazon.com, or NOOK family of devices by Barnes & Noble, Inc. of New York City, N.Y.

In some embodiments, the communications device 102 includes a combination of devices, e.g. a smartphone combined with a digital audio player or portable media player. For example, one of these embodiments is a smartphone, e.g. the IPHONE family of smartphones manufactured by Apple, Inc.; a Samsung GALAXY family of smartphones manufactured by Samsung, Inc.; or a Motorola DROID family of smartphones. In yet another embodiment, the communications device 102 is a laptop or desktop computer equipped with a web browser and a microphone and speaker system, e.g. a telephony headset. In these embodiments, the communications devices 102 are web-enabled and can receive and initiate phone calls. In some embodiments, a laptop or desktop computer is also equipped with a webcam or other video capture device that enables video chat and video call.

In some embodiments, the status of one or more machines 102, 106 in the network 104 are monitored, generally as part of network management. In one of these embodiments, the status of a machine may include an identification of load information (e.g., the number of processes on the machine, CPU and memory utilization), of port information (e.g., the number of available communication ports and the port addresses), or of session status (e.g., the duration and type of processes, and whether a process is active or idle). In another of these embodiments, this information may be identified by a plurality of metrics, and the plurality of metrics can be applied at least in part towards decisions in load distribution, network traffic management, and network failure recovery as well as any aspects of operations of the present solution described herein. Aspects of the operating environments and components described above will become apparent in the context of the systems and methods disclosed herein.

B. Methods for Identifying Mutations in Cell-Free DNA

cfDNA encompasses all DNA fragments circulating in the blood, which can be isolated from the plasma component. In cancer subjects, some of these fragments come from cancer cells (i.e., circulating tumor DNA, or ctDNA), providing a window into the somatic, or acquired, mutations in their tumor(s).

Somatic mutation calling differs from germline mutation calling in that the fraction of DNA molecules harboring a mutation can vary widely due to tumor heterogeneity and chromosomal gains and losses. This challenge is compounded when trying to identify tumor mutations in cfDNA, as the fraction of tumor-derived DNA can be extremely low (˜0.1%). Consequently, the mutation fractions in cfDNA are often lower than those observed in tissue samples from the same subject and may approach the noise levels of next-generation sequencing workflows. This can make it impossible to distinguish true somatic mutations from artifacts. Effective somatic mutation calling from cfDNA, particularly for early-stage cancer subjects, requires suppressing errors introduced in sample preparation and sequencing.

Workflow

The workflow includes a wet lab process and data processing. The wet lab process includes collecting blood or body fluids (including, but not limited to, serum, plasma, sweat, tears, urine, saliva, synovial fluid, lymphatic fluid, ascites fluid, amniotic fluid, cerebrospinal fluid, or interstitial fluid) from a subject (which may be, e.g., a known cancer subject or an asymptomatic subject that may be at risk for y cancer). Additionally or alternatively, in some embodiments, the subject suffers from or is at risk for any form of cancer, including ovarian cancer, breast cancer, colorectal cancer, lung cancer, prostate cancer, gastric cancer, pancreatic cancer, cervical cancer, liver cancer, bladder cancer, cancer of the urinary tract, thyroid cancer, renal cancer, carcinoma, melanoma, head and neck cancer, brain cancer, pediatric cancers. The blood or bodily fluids can be processed to extract cfDNA using any method known in the art. For example, the blood of the subject can be subjected to 2-spin centrifugation to isolate plasma and leukocytes (or white blood cells (WBC)). cfDNA is extracted from the non-cellular portion of the centrifuged body fluid. In addition, WBC DNA may be extracted from the white blood cells. In instances where the cfDNA is extracted from non-blood body fluids, the WBC DNA may be extracted from a separate blood draw from the subject. The cfDNA and optionally the WBC DNA are input to an assay.

The wet lab process may include whole genome library generation from the input DNA through several enzymatic steps, including end repair, A-base addition, ligation of sequencing adaptors, that is optionally followed by PCR amplification, and purification. An exemplary process has been described in the literature (Cheng et al J Mol Diagn. 17(3):251-64 (2015)). In some embodiments, the library generation process may involve the addition of unique molecular indexes (UMI) to the starting DNA molecules to improve the accuracy of mutation detection. In addition, PCR techniques can be used to include sample barcodes on each end of the cfDNA and/or WBC DNAs. In one or more embodiments, the sample barcodes may include at least one PCR primer binding site, at least one sequencing primer binding site, or any combination thereof. In one or more embodiments, the sample barcode sequence may comprise 2-20 nucleotides. In one or more embodiments, the amplified library may be followed by hybridization capture using probes, or baits, targeting specific genomic regions, as previously described in the literature (Cheng et al J Mol Diagn. 17(3):251-64 (2015)) as an example.

cfDNAs and optionally WBC DNAs associated with the same subject can be assigned unique sample barcodes. In this manner, subject specific analysis of the cfDNA and optionally WBC DNA can be carried out. The process of adding sample barcodes to the cfDNA and the optional WBC DNA is known as multiplexing. This allows large numbers of libraries to be pooled and sequenced simultaneously during a single sequencing run. With multiplexed libraries, unique sample barcode sequences are incorporated via PCR to each DNA molecule during library preparation so that each sequence read can be identified and sorted. Sequencing reads are then sorted according to their sample barcodes (i.e., the sequence reads are assigned to a given subject sample) using a computational process called de-multiplexing, allowing for proper alignment. However, such multiplex approaches come with a risk of sample misidentification due to sample barcode mis-assignment, according to Kircher M et al., Nucleic Acids Res. 2513-2524 (2012). Incorrect assignment of sequencing reads may lead to misalignment of reads or incorrect assumptions in downstream analysis. Possible causes for incorrect sample barcode assignment are sample barcode contamination, sample barcode hopping during PCR or NGS.

Many next generation sequencing-based techniques rely upon a PCR amplification step to increase the concentration of the library generated from the DNA sample prior to next-generation sequencing. Following alignment to the genome, PCR duplicates are generally identified and removed as there are inherent biases in the amplification step as some sequences become overrepresented in the final library compared to their actual abundance within the DNA sample obtained from a subject. In some next generation sequencing-based techniques, the Picard software (Broad Institute, Cambridge Mass.) is used to identify and remove PCR duplicates using their genomic coordinates.

The PCR copies of the cfDNA can be provided to a next-generation (NG) sequencing device such as, for example, an Illumina sequencer, a Lymphotrac sequencer, an Ion Torrent sequencer, and a 454 pyro-sequencer. The NG sequencer can provide raw genomic data to a genomic data processing system (such as the genomic data processing system 120, FIG. 1C). In particular, the NG sequencer can provide genomic data derived from biological samples including copies of the cfDNA and the optional WBC DNA associated with one or more subjects.

The mutation identification process 300 can be executed by the genomic data processing system 120 shown in FIG. 1C. The genomic data processing system can include or execute on one or more processors and can include scripts, modules, or computer-executable code, which when executed by one or more processors, can cause the genomic data processing system 120 to perform the process 300. The process 300 includes de-multiplexing the DNA sequence reads received from the NGS (302). De-multiplexing the DNA sequence reads can include sorting the sequence reads to their respective samples (or unique identity). The de-multiplexing of the DNA sequence reads can be applied to both the cfDNA sequence reads and the optional WBC DNA sequence reads, resulting in sorted cfDNA sequence reads associated with the same sample barcodes as well as sorted WBC DNAs sequence reads associated with the same sample barcodes. Mutation and indel calling may be performed with or without a matched normal using different mutation caller, including but not limited to MuTect, SomaticIndelDetector, and VarDict as previously described in Cheng et al J Mol Diagn. 17(3):251-64 (2015). (See, e.g., Cibulskis et al., Nat Biotechnol. 2013; 31:213-219, McKenna et al, Genome Res. 2010; 20:1297-1303, and Markovets et al., Nucleic Acids Research 2016; 44(11):e108-e.)

C. Computer Complemented Method for Distinguishing Fragments of Tumor Origin from CH-Mutation Fragments in Cell-Free DNA

Example embodiments relate to a method to discriminate between a pathogenic mutation (e.g., derived from a tumor) and mutation derived of clonal hematopoietic (CH) origin by analyzing the fragment size of cell-free DNA. Although cfDNA profiling is used in modern cancer care, a substantial amount of mutations found in plasma is attributable to CH and may be mistaken as originating from the tumor. Such CH mutations are often found in cancer driver genes that have therapeutic implications and may be mistaken as circulating tumor DNA, thus adversely affecting clinical decisions. Currently, the only way to distinguish between CH and tumor mutations is to concurrently analyze plasma and the matched white blood cell of a given individual, which is not a routine clinical practice in most commercially and clinically available cell-free DNA test.

As the defining characteristics of CH-derived cfDNA fragments as distinguished from tumor-derived cfDNA fragments are not well understood, and may differ for different tumors, etc., a computational approach for enhancing the detection of tumor mutations is presented. In various embodiments, fragment size analysis is used to distinguish CH-derived mutations from tumor-derived mutations. With more accurate assessments of whether mutations in patients' cfDNA are tumor mutations as opposed to CH mutations, a system may more effectively evaluate and characterize tumors, and recommend more suitable treatment protocols.

The data processing methods of the present disclosure are useful for enhancing the sensitivity and specificity of detecting tumor DNA fragments in cell-free DNA sample that also includes DNA fragments with CH mutations.

The present disclosure demonstrates that the size profile of cfDNA fragments bearing CH mutations are assumed to be more similar to the profile of normal white blood cells than to the profile of circulating tumor DNA, a difference that allows for discrimination between the two types of mutations in cell-free DNA. A demonstrative example studied data from patients with known CH mutations (previously identified in the white blood cells) and known tumor mutations (previously identified in the tumor), and studied the size of the fragments bearing these two groups of mutations in the corresponding plasma cell-free DNA sample. More specifically, the demonstrative example studied 44 patients with solid tumors (including prostate, bladder, breast, melanoma and lung cancers) with CH mutations previously identified by matched tumor:normal analysis using the MSK-IMPACT clinical test (see Cheng et al., J Mol Diagn 2015; 17:251-64). Blood samples were processed to extract cfDNA (see Shukla et al., JCO Precis Oncol 2017), and subjected to the MSK-IMPACT hybridization capture protocol as described except modified to adjust the adapter concentration to 4.5 μM (see Cheng et al., J Mol Diagn 2015; 17:251-64). Captured DNA libraries were sequenced on a HiSeq 4000 with PE100 reads to a mean of 646× coverage per sample, demultiplexed and aligned. CH-derived and tumor-derived nonsynonymous mutations from the tumor:normal MSK-IMPACT data were genotyped in the matched cfDNA.

In the cohort, 38 patients had 69 CH-derived mutations and 42 patients had 349 tumor-derived mutations. The demonstrative study detected a total of 63 CH-derived mutations (variant allele frequency (VAF) median 3.85%, range 0.1%-39.3%) and 169 tumor-derived mutations (VAF median 4%, range 0.1%-80%) in the matched cfDNA. Fragments bearing either tumor-derived mutations or CH-derived mutations were extracted from aligned files, resulting in 13,353 CH mutant reads, 25,373 tumor mutant reads, and 429,769 wild-type reads, aggregated across multiple loci in each group. Fragment lengths were extracted in the range of one to 720 base pairs (bp), tallied, and counts were normalized into proportions. The study then computed the difference between fragment length proportions of tumor-derived and CH fragments to highlight regions of differential enrichment, which approximately followed the ˜160 bp periodic nucleosomal pattern. This allowed the study to define two predominantly tumor-specific ROIs (127 bp to 141 bp and 272 bp to 292 bp, inclusive) and two CH-specific ROIs (173 bp to 191 bp and 346 bp to 361 bp, inclusive). For each mutation, whether tumor or CH, the study computed the proportion of fragments falling in the two tumor regions out of all fragments falling in the four selected regions, and performed classification by considering all mutations with fewer than 4 supporting reads across the selected regions were removed. Classification based on this statistic achieved an area under the curve (AUC) of 0.74. However, performance improved when the study considered mutations with at least 20 supporting reads (AUC 0.81), because estimation of the statistic from fewer reads reduced accuracy. Doing so reduced the number of mutations to 125 from 232 (54%); of these, 35 were CH mutations. As the threshold was increased further in this demonstrative example, performance on this dataset plateaued. The data in this study indicated that tumor-derived cfDNA presented a shorter fragment size distribution than CH-derived cfDNA, supporting a strategy to distinguish CH-derived mutations from tumor-derived mutation in plasma cfDNA using fragment size analysis.

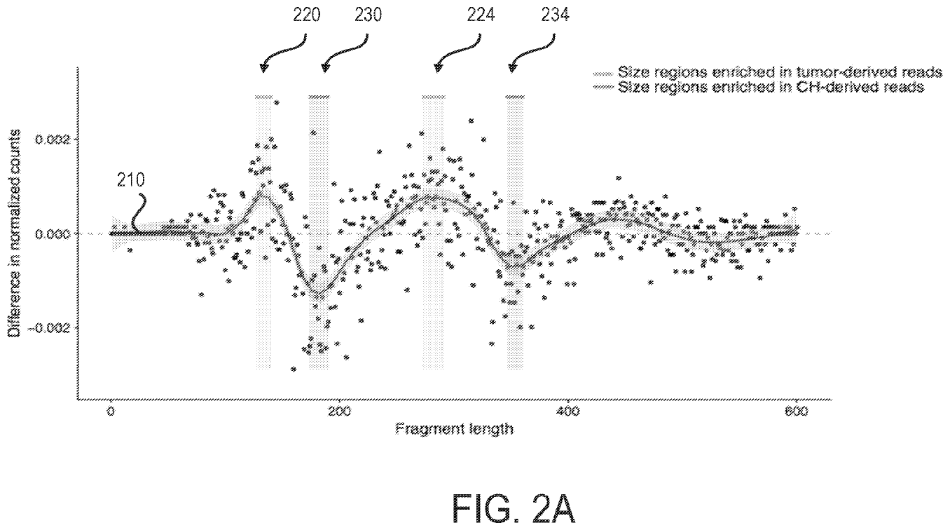

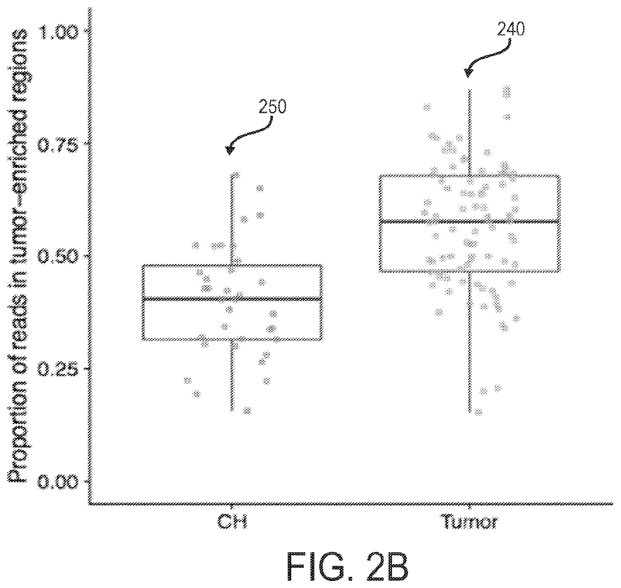

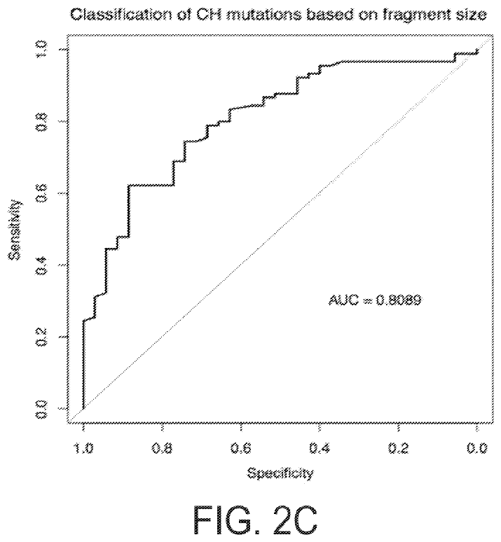

Various embodiments relate to a method to distinguish mutation derived from a tumor from mutation derived of clonal hematopoietic (CH) origin by analyzing the fragment size of cfDNA. Referring to FIG. 2A, to identify regions of the fragment size profile that may distinguish between the two groups, the method may involve computing the difference between the size distribution of tumor fragments and that of CH fragments. This may highlight one or more regions of interest (ROIs) per group per nucleosomal peak, reducing in intensity after the first peak.

In various embodiments, the method involves considering only fragments having the lengths in the ROIs, highlighted in FIG. 2A. In particular, FIG. 2A provides relative enrichment between tumor (positive values) and CH fragments (negative values), obtained by subtracting the normalized CH size profile from the normalized tumor profile. Line 210 represents a locally estimated scatterplot smoothing (LOESS) fit for the data. The highlighted rectangular areas denote the selected ROIs, with 220 and 524 corresponding with size regions enriched in tumor-derived reads, and 230 and 534 corresponding to size regions enriched in CH-derived reads.

In various embodiments, to define the ROIs, the method may use a computing system (e.g., genomic data processing system 120) to determine the distribution (probability mass function) of fragment lengths for each of the two categories (CH or tumor). They system may subtract the distribution of CH fragment lengths from that of tumor fragment lengths. The result of such an operation is depicted in FIG. 2A. The system may fit a LOESS curve to the cloud of points to highlight their trend, and select the two largest y values (e.g., two maximums) and two smallest y values (e.g., two minimums) of the curve. The system may use these selected values (e.g., the extrema) as the centers of the ROIs. In FIG. 2A, positive values represent tumor ROIs (i.e., the probability of observing a fragment of the corresponding length is higher in the tumor category); negative values are CH ROIs (i.e., the probability mass for fragments of that length is greater in the CH category). The system may define the ROI by taking a window of size w around each ROI center, where w is greater than or equal to zero.

In example embodiments, the method considers, for each mutation, supporting fragments (i.e., cfDNA fragments that bear the particular mutation) having the selected lengths (i.e., lengths within the selected ROIs). Out of all cfDNA fragments, various embodiments focus on cfDNA fragments bearing each mutation (i.e., supporting fragments), and those fragments are filtered further, such that fragments with lengths outside the intervals defined by the ROIs are ignored. The system may determine a metric, such as the proportion of fragments in tumor-enriched ROIs. The metric may be computed for each mutation under scrutiny. In example embodiments, the metric may be the number of fragments supporting the mutation with lengths in the tumor ROIs divided by the total number of fragments supporting that mutation with lengths in any of the ROIs (i.e., all fragments supporting the mutation with lengths falling in any of the defined ROIs—which in the example represented in FIG. 2A includes two tumor ROIs and 2 CH ROIs—are considered). This metric may have a range of values, such as values between 0 and 1 in the example of FIG. 2B. In this example, the metric has higher values when the mutation is of tumor origin (240), and lower values when the mutation is of CH origin (250). It should be noted that the greater the number of fragments falling in the ROIs for a particular mutation, the more precise the metric. In various embodiments, other metrics may additionally or alternatively be used. In various example embodiments, a threshold on the (minimum) number of supporting fragments may be selected, such as 2, 5, 10, 15, 20, 25, 30, or more supporting fragments.

By selecting a threshold for the metric, mutations may be classified as tumor or CH-derived according to whether the metric is above or below the threshold. In various embodiments, the threshold may be varied to achieve a performance such as the one shown in FIG. 2C for the example dataset. FIG. 2C indicates that a classification performance based on the metric represented in FIG. 2B has an Area Under the Curve (AUC) of 0.81.

In a demonstrative example, suppose five cfDNA fragments are found bearing a mutation (e.g., KRAS), with the five cfDNA fragments having lengths, for example, of 100 bp, 120 bp, 140 bp, 150 bp, and 165 bp. Also suppose the two tumor ROIs are 127 bp to 140 bp and 272 bp to 292 bp, and the two CH ROIs are 173 bp to 191 bp and 346 bp to 361 bp. In this example, the KRAS mutation has five supporting fragments, one of which falls in a tumor ROI (140 bp), and none falling in CH ROIs. The metric may be computed as the number of fragments in tumor ROIs (i.e., 1) divided by the sum of the number of fragments in tumor ROIs (i.e., 1) and the number of fragments in CH ROIs (i.e., zero): that is, 1/(1+0)=1. This is the value of the metric for this mutation.

In various embodiments, the disclosed approach involving the identified ROIs is a predictive model for determining whether a mutation is of tumor origin or CH origin. In certain embodiments, the predictive model may employ one or more machine learning techniques to distinguish between mutations having different origins. For example, a classifier or other predictive machine learning model may be trained (e.g., via supervised, semi-supervised, or unsupervised learning) using data on cfDNA of patients with known tumor and CH mutations (such as a metric based on proportions of fragments in tumor and CH ROIs), and the trained model applied to data on cfDNA of patients not known to have mutations of tumor origin.

It is noted that in alternative embodiments, this approach may be used to discriminate between two categories of nucleic acid fragments based on their fragment size using cancer-independent pathological ROIs. For example, if a sequence has a different fragmentation pattern from those of hematopoietic cells, and it is of interest to determine the origin of the fragments, one could define ROIs in a similar manner. The ROIs are thus those regions of the fragmentation size profile that are most informative of each category.

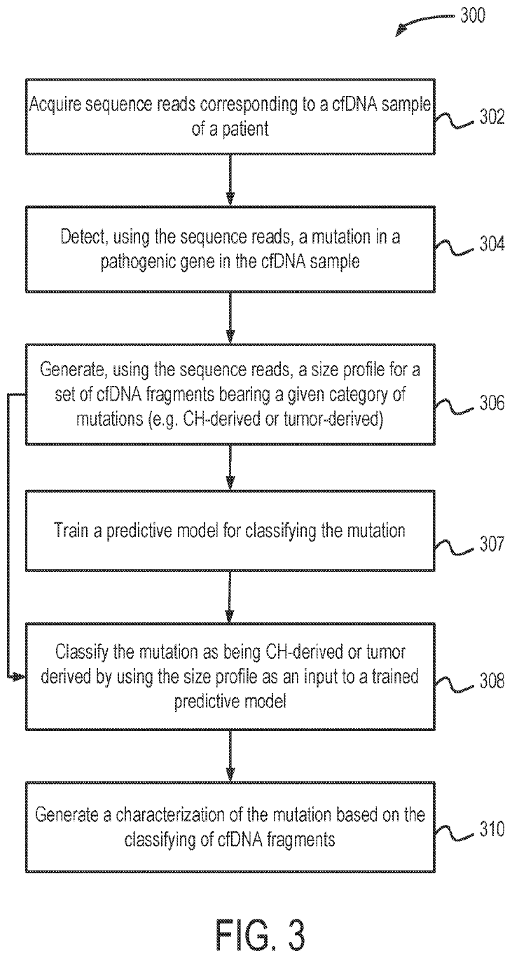

FIG. 3 illustrates a flow diagram of an example process 300 for identifying the origin of cfDNA fragments with a cancer-driving mutation. In particular, the origin identification process 300 can be executed by the genomic data processing system 120 shown in FIG. 1C. The genomic data processing system 120 can include or execute on one or more processors and can include scripts, modules, or computer-executable code, which when executed by one or more processors, can cause the genomic data processing system 120 to perform the process 300. The process 300 may be a computer-implemented method to distinguish tumor-derived (or other pathogenic) mutations from clonal hematopoietic derived mutations in cfDNA. Process 300 may include acquiring, from a sequencing device, sequence reads corresponding to cfDNA fragments in a sample of a patient (302). The patient may require screening for cancer. Process 300 may also include using the sequence reads to detect a mutation in a cancer-driving gene in the cfDNA sample of the patient (304). Detection of the mutation does not by itself reveal whether the mutation is of tumor origin or of CH origin (i.e., a true positive mutation could be of either CH or tumor origin, a misinterpretation of CH-derived mutations as tumor-derived mutation may be considered “noise” to the mutation detection process). Process 300 may additionally include generating, using the sequence reads, a size profile for a set of cfDNA fragments with the mutation (306). The size profiles indicate a number of cfDNA fragments (frequency) for each fragment length (in, e.g., number of base pairs). As used here, these “size profiles” are for fragments bearing the particular mutation, rather than all cfDNA fragments bearing tumor mutations. Process 300 may moreover include classifying certain cfDNA fragments (based on fragment size) as having a tumor-derived mutation and certain other cfDNA fragments (based on fragment size) as having a CH-derived mutation. The DNA fragments may be classified by feeding the size profile to a predictive model for the mutation (i.e., a mutation-specific predictive model) (308).

In various embodiments, if the predictive model to be applied is not already trained, process 300 may include training a predictive model (307) for categories of mutations under investigation (e.g., CH vs tumor). The predictive model may be trained, for example, based on a determination of the distribution of fragment sizes for each category in advance on a large data set. In various embodiments, the predictive model may be trained before step 308 (or before starting process 300), if not previously trained, for use in classification. Each new analysis, where the predictive model to be applied is available, step 307 may be omitted, and process 300 may proceed to apply the predictive model (e.g., may proceed directly to classifying (308) called mutations (304) on the basis of previously determined distributions).

In various embodiments, the system 120 may train the predictive model using sequence reads corresponding to cfDNA fragments in samples of a plurality of training subjects with known tumor mutations and/or known CH mutations. Using the sequence reads, the system may generate a tumor fragment size profile and a CH fragment size profile. The system 120 may apply a smoothing operation (e.g., LOESS) to the size profiles to obtain a trend line. System 120 may define a tumor ROI and a CH ROI in the trend line. In certain embodiments, the system 120 may compute a difference between the tumor fragment size profile and the CH fragment size profile, and obtain the trend line by applying the smoothing operation to the difference. The trend line may include a set of one or more extrema, such as a set comprising a first extremum (e.g., a first maximum or minimum) and a second extremum (e.g., a second maximum or minimum), with the tumor ROI centered about (or terminating in or otherwise comprising) the first extremum and the CH ROI centered about (or terminating in or otherwise comprising) the second extremum. The tumor ROI may be a first number of base pairs on one side of the first extremum (e.g., the tumor ROI may terminate at the first extremum) or on both sides of the first extremum (e.g., the first extremum is between the beginning and end of the tumor ROI, such as midway between the beginning and end), and the CH ROI may be a second number of base pairs on one side of the second extremum (e.g., the CH ROI may terminate at the second extremum), or on both sides of the second extremum (e.g., the second extremum is between the beginning and end of the CH ROI, such as midway between the beginning and end). In various embodiments, multiple tumor ROIs and/or multiple CH ROIs may be centered about (or may terminate in or otherwise comprise) various extrema in the set of extrema. For example, in example embodiments, a first tumor ROI may comprise (e.g., may be centered about) a first extremum (e.g., a first maximum or minimum), a first CH ROI may comprise (e.g., may be centered about) a second extremum (e.g., a second maximum or minimum), a second tumor ROI may comprise (e.g., may be centered about) a third extremum (e.g., a third maximum or minimum), and a second CH ROI may comprise (e.g., may be centered about) a fourth extremum (e.g., a fourth maximum or minimum), and so forth.

The system 120 may further generate the predictive model by generating, for each mutation, a standard metric based on the proportion of fragments in the tumor and CH ROIs. The standard metric may be a number of cfDNA fragments with lengths in the tumor ROI divided by a total number of DNA fragments with lengths in both the tumor ROI and the CH ROI. A metric threshold may be selected for use in classifying DNA fragments as having the tumor-derived mutation or the CH-derived mutation. Process 300 may further include generating a characterization of the mutation based on the classifying of cfDNA fragments (310). Characterizations may identify or relate to a specific quantity or quality of the gene mutation, the cfDNA sample, or the patient, such as mutation origin.

In various embodiments, the predictive model may be based on the tumor fragment size profile and the CH fragment size profile. In some embodiments, the predictive model may be trained on the tumor fragment size profile and the CH fragment size profile using supervised, semi-supervised, and/or unsupervised learning.

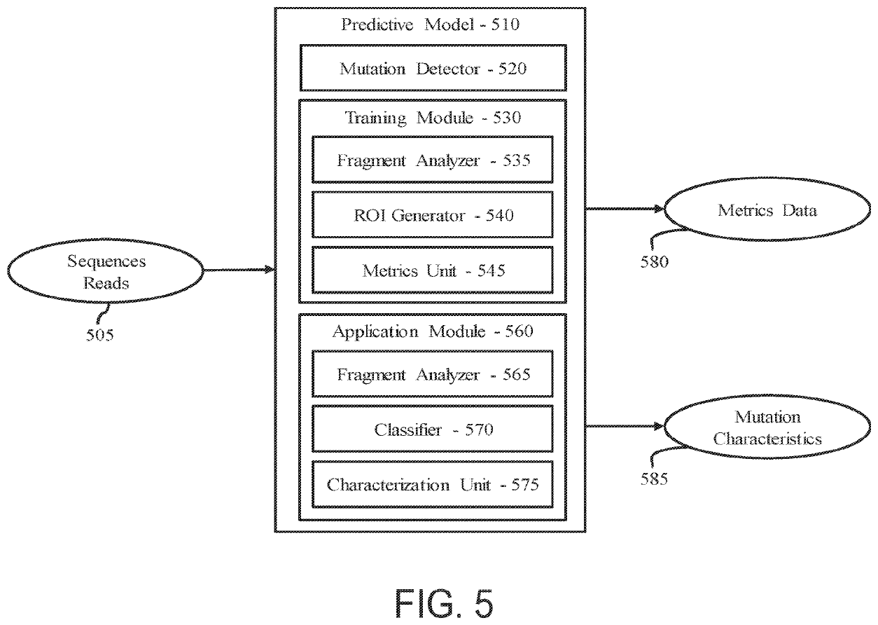

An example system 500 comprising a predictive modeler 510 is depicted in FIG. 5. The predictive modeler 510 may be implemented by or via the genomic data processing system 120. Predictive modeler 510 may receive sequence reads 505 acquired via one or more sequencers. The sequence reads may correspond to cfDNA fragments in samples of subjects (for training purposes) or patients (for validation or application purposes). The predictive modeler 510 may comprise a mutation detector 520 configured to detect, from the sequence reads 505, a gene mutation in the fragments in the cfDNA sample. The predictive modeler 510 also comprises a training module 530 configured to train a model using cfDNA samples from training subjects, and an application module 560 for applying the trained model to cfDNA samples from patients, for classifying and characterizing mutations. The predictive modeler 510 may output metrics data 580 (e.g., metric values and metric thresholds) as well as mutation characteristics 585 (such as the origin of a gene mutation detected in fragments in a cfDNA sample of a patient). System 500 may output metrics 580 and/or characterizations 585 on one or more user computing devices (e.g., visually on a display screen or otherwise).

In various embodiments, the training module 530 comprises a fragment analyzer 535, which is configured to accept sequence reads corresponding to cfDNA fragments in samples of a plurality of subjects with known tumor mutations and/or known CH mutations. The fragment analyzer 535 may generate, for fragments in the cfDNA sample, size profiles comprising the number of fragments for each fragment size (e.g., the number of fragments versus the number of base pairs). The fragment analyzer 535 may generate a tumor fragment size profile corresponding to cfDNA fragments of tumor origin, and a CH fragment size profile corresponding to cfDNA fragments with CH origin.

The training module 530 comprises an ROI generator 540 configured to generate ROIs for fragment sizes corresponding to tumor and CH origins. To identify ROIs, the ROI generator 540 may be configured to generate a trend line for the size profiles from the fragment analyzer 535. The trend line may be obtained by applying a smoothing operation (e.g., LOESS) to size profiles. The ROI generator 540 may determine a difference between the tumor fragment size profile and the CH fragment size profile, and apply the smoothing operation to the difference to obtain the trend line. The trend line may comprise a set of extrema with one or more maximums and one or more minimums. One or more tumor ROIs and one or more CH ROIs may be defined based on the extrema, with each ROI centered about a maximum or a minimum in the set of extrema. For example, each ROI may be defined by taking a window of size w (e.g., a window of a certain number of base pairs, such as five, in at least one direction) around each ROI center, or the ROI center without a window. Each ROI may terminate in, or comprise within it (at its center or otherwise), a corresponding extremum.

The training module 530 may comprise a metrics unit 545, which may be configured to generate one or more standard metrics based on a proportion of fragments in the tumor or CH ROIs. This standard metric may have a value ranging from zero to one. A different standard metric may be generated for each gene mutation. The standard metric may be a number of cfDNA fragments with lengths in the one or more tumor ROIs divided by a total number of cfDNA fragments with lengths in both the one or more tumor ROIs and the one or more CH ROIs. In such a case, the standard metric would be higher when the mutation is of tumor origin, or lower when the mutation is of CH origin. In various embodiments, the standard metric for each mutation may be a number of cfDNA fragments with lengths in the one or more CH ROIs divided by a total number of cfDNA fragments with lengths in both the one or more tumor ROIs and the one or more CH ROIs. In such a case, the standard metric would be higher when the mutation is of CH origin, or lower when the mutation is of tumor origin. The metrics unit 545 may select a metric threshold based on, for example, the performance of a dataset with the sequence reads obtained from the subjects (see, e.g., FIG. 2C) so as to balance sensitivity and specificity.

The predictive modeler 510 may also comprise an application module 560 with a fragment analyzer 565 configured to accept sequence reads corresponding to cfDNA fragments in a sample of a patient (i.e., a test subject). The mutation detector 520 may identify, in the sequence reads from the patient's sample, a gene mutation for which a standard metric is generated by the metrics unit 545 of the training module 530. The application module 560 may comprise a fragment analyzer 565 configured to generate, for fragments in the cfDNA sample of the patient, size profiles comprising the number of fragments for each fragment size (e.g., the number of fragments versus the number of base pairs).

The classifier 570 may use the size profiles from the fragment analyzer 565 to classify cfDNA fragments as having tumor origin or CH origin. For example, the classifier 570 may, using the tumor and CH ROIs from the ROI generator 540, determine subsets of cfDNA fragments as having tumor origin or CH origin based on whether the fragment's length falls in a tumor ROI or a CH ROI, respectively. The classifier 570 may also generate metrics from the cfDNA fragments bearing the mutation. For example, the classifier 570 may determine a patient metric for the classified cfDNA fragments, such as a proportion of cfDNA fragments that fall in ROIs for a particular origin as a fraction of all cfDNA fragments falling in any of the ROIs identified by the ROI generator 540. The characterization unit 575 may then characterize the cfDNA fragments in the patient's sample using metrics from classifier 570 and the metrics unit 545. For example, the patient metric (from classifier 570) may be compared with the metric threshold (from metrics unit 545) corresponding to the mutation detected in the patient's cfDNA sample. The characterization unit 575 may determine, for example, that the gene mutation detected in the patient's cfDNA has a tumor origin if the patient metric is equal to or greater than the metric threshold, and that the gene mutation has a CH origin if the patient metric is below the metric threshold.

In various embodiments, the training module 530 may output (e.g., as metrics data 580), for example, a set of ROIs per category (e.g., a set of tumor ROIs and a set of CH ROIs), a set of metrics (e.g., the standard metric), and/or a threshold or other discriminator to enable classification (e.g., the metric threshold). In various embodiments, the application module 560 may output (e.g., as mutation characteristics 585), for example, a predicted category for every input mutation, and/or a degree of confidence in the classification (e.g. a probability or a score).

In various embodiments, the metrics unit 545, the classifier 570, and/or the characterization unit 575, for use in performing classification, may accept clinical covariates (e.g., patient sex and age), and potentially other features that may be derivable from the sequence reads.

In various embodiments, the predictive modeler 510 (e.g., via training module 530 and application module 560) learns ROIs, generates one or more metrics per gene mutation, and performs classification based on the metrics. To learn various parameters, the predictive modeler 510 (e.g., via the training module 530) may use data in a “training set” and data in a “test set”: parameters may be proposed based on the training data, and performance of the parameters may be assessed using the test data. This approach to learning parameters in a machine learning environment is useful for assessing the generalizability of the predictive modeler 510 (i.e., its performance on new datasets).

In various embodiments, the metric threshold may be chosen based on the performance of the trained model on a test set. In various other embodiments, the metric threshold could be set according to clinical needs, for example, to maximize sensitivity (e.g., characterize mutations as tumor mutations unless confidence is very high that the mutations are CH mutations) or specificity (e.g., characterize mutations as tumor mutations only if confidence is very high that the mutations are tumor mutations).

Various embodiments include a process of “training” a predictive model. Such a training process may use a dataset in which the identity (e.g., CH or tumor) of gene mutations (or at least a subset of the gene mutations) is known, and the fragment lengths are available. Any form of learning (e.g., discriminative or generative) that enable distinguishing between the two categories may be utilized.

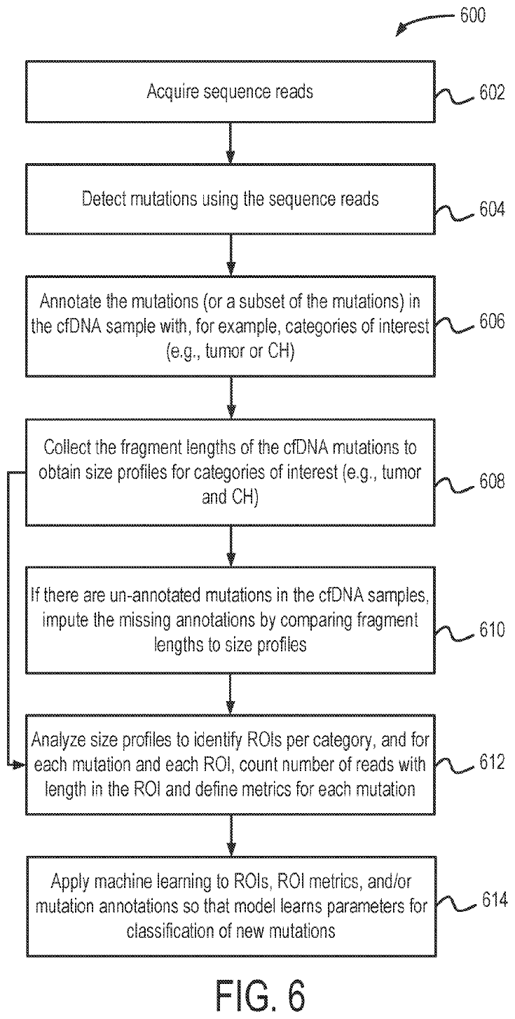

Referring to FIG. 6, in various embodiments, the steps in a training process 600 (which may be implemented via training module 530) may include, for example: (602) acquiring sequence reads from one or more cfDNA samples, acquiring sequence reads from one or more matched (that is coming from the same patient) buffy coat samples, and/or acquiring sequence reads from one or more matched tumor samples; (604) detecting, using the sequence reads, mutations in the cfDNA samples (where the mutations need not be in pathogenic genes), detecting, using the sequence reads, mutations in the buffy coat samples, and/or detecting, using the sequence reads, mutations in the tumor samples; (606) annotating the mutations (or a subset of the mutations) in the cfDNA sample as coming from, for example, the “tumor” (if they are also found in the matched tumor sample(s) but not in the matched buffy coat sample(s)) or “CH” (if they are also found in the matched buffy coat sample(s) but not in the matched tumor sample(s)); (608) collecting the fragment lengths of the cfDNA mutations annotated as, for example, CH (to obtain a CH size profile), and/or collecting the fragment lengths of the cfDNA mutations annotated as tumor (to obtain a tumor size profile); optionally, (610) if it was not possible to annotate all mutations in the cfDNA samples, the process may include imputing the missing annotations by comparing their fragment lengths to the CH size profile and the tumor size profile; multiple imputation methods may be applicable here; (612) analyzing the two size profiles (e.g., CH and tumor) to identify a set of ROIs per category (e.g., CH or tumor), and for each mutation and each ROI, counting the number of reads with length included in the ROI and computing function(s) of these counts (thereby defining a set of metrics for each mutation); and (614) supplying the ROIs, the mutation-specific ROI metrics, and mutation annotations (e.g., CH or tumor mutation) to one or more machine learning processes (e.g., logistic regression, support vector machines, neural networks, random forests, K-nearest neighbors, probabilistic models, Bayesian classifiers, and/or others), where the one or more processes learn a set of parameters, such as a threshold that allows for classification of new mutations on the basis of their fragment lengths (e.g., via the ROIs).

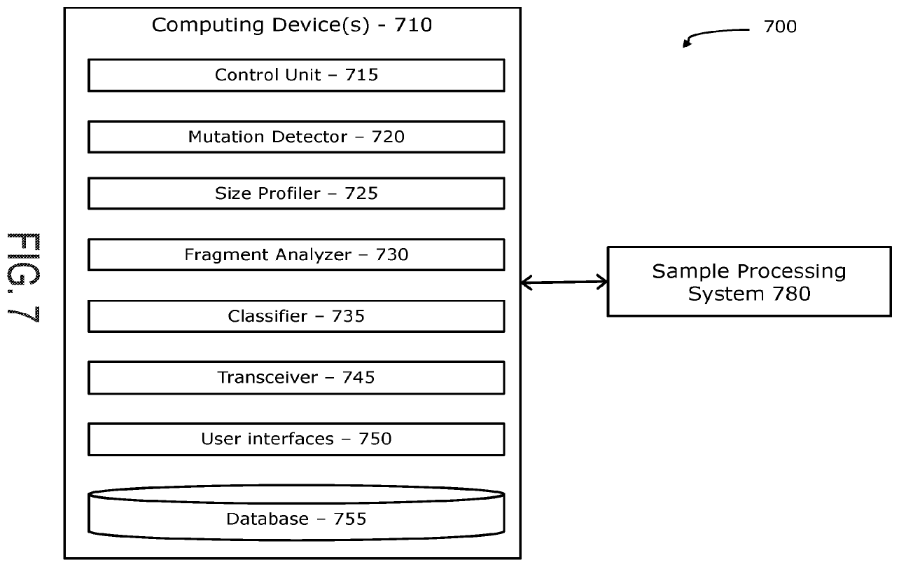

Referring to FIG. 7, in various potential embodiments, a system 700 may include a computing device 710 (or multiple computing devices, co-located or remote to each other) and a sample processing system 780. In various embodiments, computing device 710 (or components thereof) may be integrated with the sample processing system 780 (or components thereof). Components of computing device 710 may be implemented using a combination of computing hardware and software code. In various embodiments, the sample processing system 780 may include, may be, or may employ, a next-generation sequencer. The computing device 710 and sample processing system 780 may interface through wired or wireless communications protocols, and may communicate through various network infrastructures.

In various potential embodiments, the computing device 710 (or multiple computing devices) may be used to control, and receive signals acquired via, components of sample processing system 780. The computing device 710 may include one or more processors and one or more volatile and non-volatile memories for storing computing code and data that are captured, acquired, recorded, and/or generated. The computing device 710 may include a control unit 715 that is configured to exchange control signals with sample processing system 780, allowing the computing device 710 to be used to control, for example, processing of samples and/or delivery of data generated and/or acquired through processing of samples. For example, control unit 715 may generate and transmit a signal to cause the a sequencing device of sample processing system 780 to begin processing a sample with cfDNA fragments in a sample of a test subject and generate sequence reads. The control unit 715 may then acquire, from (or via) the sample processing system 780 (e.g., a memory or database thereof, or accessible thereto), sequence reads corresponding to the cfDNA fragments in the sample. Alternatively or additionally, the sample processing system 780 may provide sequence reads when available without being requested

In various potential embodiments, a mutation detector 720 may be used, for example, to perform analyses of data captured using sample processing system 780, and may include, for example, identifying mutations in cfDNA based on sequence reads. A size profiler 725 may be used to generate a size profile for a set of cfDNA fragments with the gene mutation. The size profile may identify how many cfDNA fragments are detected for certain fragment lengths.

In various potential embodiments, the size profiler 725 may use sequence reads from the sequencing device to generate a tumor fragment size profile and a CH fragment size profile. A fragment analyzer 730 may perform various analyses on fragment size profiles. For example, the fragment analyzer 730 may determine a difference between the tumor fragment size profile and the CH fragment size profile. The fragment analyzer 730 may apply smoothing operations to the difference between the tumor fragment size profile and the CH fragment size profile to obtain a trend line that includes various extrema, such as a first set of extrema and a second set of extrema. The fragment analyzer may define one or more tumor ROIs centered about extrema in the first set of extrema, and one or more CH ROIs centered about extrema in the second set of extrema. The fragment analyzer may generate, for each mutation, a metric based on the proportion of fragments in the tumor and CH ROIs. The metric may be a number of cfDNA fragments with lengths in one of the tumor ROIs or the CH ROIs, divided by a total number of cfDNA fragments with lengths in both the tumor ROIs and the CH ROIs.

In various potential embodiments, a classifier 735 may classify, in the set of cfDNA fragments in the cfDNA sample, a first subset of cfDNA fragments as having a tumor origin and a second subset of cfDNA fragments as having a CH origin. The classifier may feed the size profile as an input to a mutation-specific predictive model that is configured to generate a first set of ranges of fragment lengths for fragments with the tumor origin and a second set of ranges of fragment lengths for fragments of the CH origin. The first subset of cfDNA fragments may have lengths falling in the first set of ranges and the second subset of cfDNA fragments may have lengths falling in the second set of ranges. The classifier may additionally or alternatively be used to classify a mutation in cfDNA of a test subject as having either tumor origin or CH origin using a metric threshold from fragment analyzer 730. The classifier 735 may be a component of, or may comprise components of, predictive modeler 510. The classifier 735 (and/or predictive modeler 510) may be used to implement various machine learning functionality discussed herein, such as applying various machine learning techniques to one or more training datasets (e.g., datasets with genomic data from various cohorts) to train machine learning classifiers for various predictions or other classifications, and may employ a machine learning classifier to analyze genomic data (e.g., from one or more patients or other subjects) to make various predictions or other classifications. The classifier 735 may, based on classifications corresponding to tumor or CH origin, generate characterizations of mutations (e.g., identification of origins of gene mutations based on a comparison of the metric with a metric threshold).

In various potential embodiments, a transceiver 745 allows the computing device 710 to exchange readings, control commands, and/or other data with sample processing system 780 (or components thereof). One or more user interfaces 750 allow the computing device 710 to receive user inputs (e.g., via a keyboard, touchscreen, microphone, camera, etc.) and provide outputs (e.g., via display screen, audio speakers, etc.). The computing device 710 may additionally include one or more databases 755 (stored in, e.g., on or more computer-readable non-volatile memory devices) for storing, for example, data and analyses obtained from or via mutation detector 720, size profiler 725, fragment analyzer 730, classifier 735, and/or sample processing system 780. In some implementations, database 755 (or portions thereof) may alternatively or additionally be part of another computing device that is co-located or remote and in communication with computing device 710 and/or sample processing system 780 (or components thereof).

In various embodiments, model training may be followed by classification of mutations of unknown origin for samples of interest. In various embodiments, a classification process may include, for example: (1) acquiring sequence reads from a cfDNA sample of a patient; (2) detecting, using the sequence reads, a mutation in the cfDNA sample (where the mutations need not be in pathogenic genes); (3) collecting the lengths of the fragments bearing the mutation; (4) collecting the ROI metrics of the mutation (in certain embodiments, this step can replace the previous step); (5) feeding this information to the trained model; and (6) annotating the mutation as being, for example, CH or tumor according to the output of the trained model.

These results demonstrate that the data processing methods and systems disclosed herein are useful for detecting cancer-related mutations in cell-free DNA (cfDNA) sequence data with a high degree of accuracy and sensitivity.

It is noted that, in various embodiments, the functions performed by the systems, devices, and components depicted in, for example, FIGS. 1A-1D and 5 may be performed by a greater number of components or fewer components, and may be performed by other combinations of devices and systems. For example, the functions performed by one component as depicted may instead be performed by two or more components, and/or the functions performed by two or more components as depicted may instead be performed by one component. Similarly, functions may be redistributed among components, devices, and systems. For example, the functions performed by one combination of components, devices, and/or systems as depicted may instead be performed by another combination of components, devices, and/or systems.

The terms “region of interest” and “ROI” refer to ranges of nucleic acid fragment lengths (e.g., number of base pairs). These one or more ROIs may be defined according to various criteria specific to a particular pathology (e.g., CH, cancer, etc.) to determine an interval for a particular pathology. Examples of such criteria include the distribution (e.g., probability mass function) of nucleic acid fragment lengths for a pathology, differences between distributions of nucleic acid fragment lengths of 2 or more different pathologies, statistical properties of the distributions of nucleic acid fragment lengths of 2 or more different pathologies, and expert knowledge. In one example, the distribution of CH nucleic acid fragment lengths may be subtracted from that of tumor nucleic acid fragment lengths. A LOESS curve may be fitted to all of the points to highlight their trend, and the one or more largest and/or smallest extremes per pathology of the curve may be selected (which represent centers of the ROIs). In this particular example, positive values are tumor ROIs (the probability to observe a fragment of that length is higher in the tumor category); negative values are CH ROIs (the probability mass for fragments of that length is greater in the CH category) and the ROI may be defined by taking a window of size w (e.g., a window of at least 0 base pair in at least one direction) around each ROI center or the ROI center without a window.

The term “adapter” refers to a short, chemically synthesized, nucleic acid sequence which can be used to ligate to the end of a nucleic acid sequence in order to facilitate attachment to another molecule. The adapter can be single-stranded or double-stranded. An adapter can incorporate a short (typically less than 50 base pairs) sequence useful for PCR amplification or sequencing. In some embodiments, the adapter includes a unique molecular identifier.

The term “hold out” in the context of machine learning refers to splitting up a dataset into a ‘training set’ and ‘test set’. The training set is used to train a model, and the test set is “held out” and used to see how well that model performs on unseen data.

The terms “variant allele fraction,” “VAF,” “mutant allele fraction” or “MAF” refer to fractions of a mutant allele over the total number of mutant (alternate allele) plus wild-type alleles (reference allele).

“Unique molecular identifiers” or “UMIs” are random nucleotide sequences used to tag each DNA molecule (fragment) prior to library amplification, thereby aiding in the identification of PCR duplicates. If two reads align to the same location and have the same UMI, it is highly likely that they are PCR duplicates originating from the same DNA molecule prior to amplification. As a result, all sequence reads with identical genomic coordinates and UMIs can be collapsed into a single representative read, which is useful for obtaining an accurate estimate of the relative concentration of the DNA molecules in the DNA sample.

The term “plurality of first DNA reads” refers to DNA sequence reads that are derived from the first oligonucleotide strand (e.g., sense strand) of a double-stranded DNA molecule. In some embodiments, the plurality of first DNA reads originate from cfDNA or white blood cells (WBC).

The term “plurality of second DNA reads” refers to DNA sequence reads that are derived from the second oligonucleotide strand (e.g., anti-sense strand) of a double-stranded DNA molecule. The plurality of second DNA reads may be at least partially or completely complementary to the plurality of first DNA reads (e.g., at least 70%. 75%, 80%, 85%, 90%, or 95% complementary). In some embodiments, the plurality of second DNA reads originate from cfDNA or white blood cells (WBC). The term “white blood cells” or “WBC” refers to blood cells that are colorless, lack hemoglobin, contain a nucleus, and include lymphocytes, monocytes, neutrophils, eosinophils, and basophils.

The terms “complementary” or “complementarity” as used herein with reference to polynucleotides (i.e., a sequence of nucleotides such as an oligonucleotide or a target nucleic acid) refer to the base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence “5′-A-G-T-3′” is complementary to the sequence “3′-T-C-A-5.” Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

“Coverage” or “depth” as used herein refers to the number of reads that align to, or “cover,” known reference bases. The next-generation sequencing (NGS) coverage level often determines whether variant discovery can be made with a certain degree of confidence at particular base positions.

“Next-generation sequencing or NGS” as used herein, refers to any sequencing method that determines the nucleotide sequence of either individual nucleic acid molecules (e.g., in single molecule sequencing) or clonally expanded proxies for individual nucleic acid molecules in a high throughput parallel fashion (e.g., greater than 103, 104, 105 or more molecules are sequenced simultaneously). In one embodiment, the relative abundance of the nucleic acid species in the library can be estimated by counting the relative number of occurrences of their cognate sequences in the data generated by the sequencing experiment. Next generation sequencing methods are known in the art. Examples of Next Generation Sequencing techniques include, but are not limited to pyrosequencing, Reversible dye-terminator sequencing, SOLiD sequencing, Ion semiconductor sequencing, Sequencing by synthesis (SBS), Helioscope single molecule sequencing etc. Next generation sequencing methods can be performed using commercially available kits and instruments from companies such as the Life Technologies/Ion Torrent PGM or Proton, the Illumina HiSEQ or MiSEQ, and the Roche/454 next generation sequencing system.

As used herein, “oligonucleotide” refers to a molecule that has a sequence of nucleic acid bases on a backbone comprised mainly of identical monomer units at defined intervals. The bases are arranged on the backbone in such a way that they can bind with a nucleic acid having a sequence of bases that are complementary to the bases of the oligonucleotide. The most common oligonucleotides have a backbone of sugar phosphate units. A distinction may be made between oligodeoxyribonucleotides that do not have a hydroxyl group at the 2′ position and oligoribonucleotides that have a hydroxyl group at the 2′ position. Oligonucleotides of the method which function as primers or probes are generally at least about 10-15 nucleotides long and more preferably at least about 15 to 35 nucleotides long, although shorter or longer oligonucleotides may be used in the method. The exact size will depend on many factors, which in turn depend on the ultimate function or use of the oligonucleotide.

As used herein, a “sample” refers to a substance that is being assayed for the presence of a mutation in cfDNA, e.g., ctDNA. Processing methods to release or otherwise make available a nucleic acid for detection are well known in the art and may include steps of nucleic acid manipulation. A sample may be a body fluid. In some cases, a biological sample may consist of or comprise serum, plasma, sweat, tears, urine, saliva, synovial fluid, lymphatic fluid, ascites fluid, amniotic fluid, or interstitial fluid, cerebrospinal fluid, and the like.

The embodiments described herein have been described with reference to drawings. The drawings illustrate certain details of specific embodiments that provide the systems, methods and programs described herein. However, describing the embodiments with drawings should not be construed as imposing on the disclosure any limitations that may be present in the drawings.

It is noted that terms such as “approximately,” “substantially,” “about,” or the like may be construed, in various embodiments, to allow for insubstantial or otherwise acceptable deviations from specific values. In various embodiments, deviations of 20 percent may be considered insubstantial deviations, while in certain embodiments, deviations of 15 percent may be considered insubstantial deviations, and in other embodiments, deviations of 10 percent may be considered insubstantial deviations, and in some embodiments, deviations of 5 percent may be considered insubstantial deviations. In various embodiments, deviations may be acceptable when they achieve the intended results or advantages, or are otherwise consistent with the spirit or nature of the embodiments.

It should be noted that although the diagrams herein may show a specific order and composition of method steps, it is understood that the order of these steps may differ from what is depicted. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative embodiments. Accordingly, all such modifications are intended to be included within the scope of the present disclosure as defined in the claims. Such variations will depend on the machine-readable media and hardware systems chosen and on designer choice. It is understood that all such variations are within the scope of the disclosure. Likewise, software and web implementations of the present disclosure may be accomplished with standard programming techniques with rule based logic and other logic to accomplish the various database searching steps, correlation steps, comparison steps and decision steps.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principals of the disclosure and its practical application to enable one skilled in the art to utilize the various embodiments and with various modifications as are suited to the particular use contemplated. Other substitutions, modifications, changes and omissions may be made in the design, operating conditions and arrangement of the embodiments without departing from the scope of the present disclosure as expressed in the appended claims.

1. A computer-implemented method of employing machine learning to distinguish tumor-derived mutations from clonal hematopoietic derived mutations in cell-free DNA (cfDNA), the method comprising:
acquiring, by one or more processors, from a sequencing device, sequence reads corresponding to cfDNA fragments in a sample of a test subject;
detecting, by the one or more processors, using the sequence reads corresponding to the cfDNA fragments, a gene mutation in the cfDNA;
generating, by the one or more processors, a size profile for a set of cfDNA fragments with the gene mutation of specific origins, the size profile identifying how many cfDNA fragments are detected for each fragment length in a plurality of fragment lengths;
classifying, by the one or more processors, in the set of cfDNA fragments in the cfDNA sample, a first subset of cfDNA fragments as having a tumor origin and a second subset of cfDNA fragments as having a CH origin by feeding the size profile as an input to a mutation-specific predictive machine-learning model that is configured to generate a first set of ranges of fragment lengths for fragments with the tumor origin and a second set of ranges of fragment lengths for fragments of the CH origin, wherein the first subset of cfDNA fragments have lengths falling in the first set of ranges and the second subset of cfDNA fragments have lengths falling in the second set of ranges; and
generating, by the one or more processors, a characterization of the mutation based on the classifying of cfDNA fragments.

2. The method of claim 1, wherein the method further comprises generating a metric based on the size profile, and wherein generating the characterization comprises identifying an origin of the gene mutation based on a comparison of the metric with a metric threshold.

3. The method of claim 2, wherein the metric is a proportion of fragments in one of the subsets of cfDNA fragments to fragments in both of the subsets of cfDNA fragments.

4. The method of claim 2, wherein the predictive machine-learning model is further configured to generate the metric threshold based on an analysis of cfDNA samples of a plurality of training subjects.

5. The method of claim 1, further comprising training the predictive machine-learning model by:
acquiring, by the one or more processors, from the sequencing device, sequence reads corresponding to cfDNA fragments in samples of a plurality of subjects with known tumor mutations and/or known CH mutations; and
generating, by the one or more processors, using the sequence reads from the sequencing device, a tumor fragment size profile and a CH fragment size profile.

6. The method of claim 5, further comprising training the predictive machine-learning model by:
obtaining a trend line, wherein obtaining the trend line comprises applying, by the one or more processors, a smoothing operation to the tumor fragment size profile and the CH fragment size profile; and
defining in the trend line, by the one or more processors, one or more tumor regions of interest (ROI) and one or more CH ROIs, the tumor ROIs corresponding with the first set of ranges of fragment lengths and the CH ROIs corresponding with the second set of ranges of fragment lengths.

7. The method of claim 6, further comprising determining, by the one or more processors, a difference between the tumor fragment size profile and the CH fragment size profile, wherein obtaining the trend line comprises applying the smoothing operation to the difference to obtain the trend line.

8. The method of claim 6, wherein the predictive machine-learning model is further trained by generating, by the one or more processors, for each mutation, a metric based on the proportion of fragments in the tumor and CH ROIs.

9. The method claim 8, wherein the metric is a number of cfDNA fragments with lengths in one of the tumor ROIs or the CH ROIs, divided by a total number of cfDNA fragments with lengths in both the tumor ROIs and the CH ROIs.

10. The method of claim 8, further comprising selecting a metric threshold for use in classifying cfDNA fragments as having a tumor-derived mutation or a CH-derived mutation.

11. The method of claim 6, wherein the predictive machine-learning model is trained on a tumor fragment size profile and a CH fragment size profile.

12. The method of claim 6, wherein the trend line includes a set of one or more extrema, wherein the tumor and CH ROIs are centered about extrema in the set of extrema.

13. The method of claim 12, wherein the tumor ROI is a first number of base pairs on one or both sides of a first extremum, and wherein the CH ROI is a second number of base pairs on one or both sides of a second extremum.

14. (canceled)

15. The method of claim 1, wherein the gene mutation is in one or more cancer-related genes.

16. The method of claim 1, wherein the predictive machine-learning model is trained on a tumor fragment size profile and a CH fragment size profile using unsupervised learning.

17-18. (canceled)

19. A computing system for distinguishing tumor-derived mutations from clonal hematopoietic derived mutations in cell-free DNA (cfDNA) through machine learning, the computing system comprising one or more processors configured to:
acquire, from a sequencing device, sequence reads corresponding to cfDNA fragments in a sample of a test subject;
detect, using the sequence reads corresponding to the cfDNA fragments, a gene mutation in the cfDNA;
generate a size profile for a set of cfDNA fragments with the gene mutation, the size profile identifying how many cfDNA fragments are detected for each fragment length in a plurality of fragment lengths;
classify, in the set of cfDNA fragments in the cfDNA sample, a first subset of cfDNA fragments as having a tumor origin and a second subset of cfDNA fragments as having a CH origin by feeding the size profile as an input to a predictive machine-learning model that is configured to generate, for the gene mutation, a first set of one or more ranges of fragment lengths for fragments with the tumor origin and a second set of one or more ranges of fragment lengths for fragments of the CH origin, wherein the first subset of cfDNA fragments have lengths falling in the first set of ranges and the second subset of cfDNA fragments have lengths falling in the second set of ranges; and
generate, using a metric threshold, a characterization of the mutation based on the classifying of cfDNA fragments.

20. The computing system of claim 19, the one or more processors further configured to train the predictive machine-learning model by:
acquiring, from the sequencing device, sequence reads corresponding to cfDNA fragments in samples of a plurality of subjects with known tumor mutations and/or known CH mutations; and
generating, by the one or more processors, using the sequence reads from the sequencing device, a tumor fragment size profile and a CH fragment size profile.

21. The computing system of claim 19, the one or more processors further configured to train the predictive machine-learning model by:
obtaining a trend line by applying a smoothing operation to the tumor fragment size profile and the CH fragment size profile; and
defining in the trend line one or more tumor regions of interest (ROIs) and one or more CH ROIs, the tumor ROIs corresponding with the first set of ranges of fragment lengths and the CH ROIs corresponding with the second set of ranges of fragment lengths.

22-32. (canceled)

33. A method, comprising:
(a) extracting cell-free DNA (cfDNA) comprising tumor-origin cfDNA fragments and CH-origin cfDNA fragments from substantially cell-free samples of blood plasma and/or blood serum of a plurality of subjects;
(b) producing one or more tumor regions of interest (ROIs) and one or more CH ROIs for the cfDNA fragments of (a) by:
(i) generating a tumor fragment size profile and a CH fragment size profile;
(ii) applying a smoothing operation to a difference between the tumor fragment size profile and the CH fragment size profile to obtain a trend line with a set of extrema comprising one or more maximums and one or more minimums; and
(iii) defining the tumor and CH ROIs as sets of ranges of cfDNA fragment sizes based on the maximums and minimums; and
(c) extracting and analyzing cfDNA fragments in a sample of a patient using the tumor and CH ROIs.

34. The method of claim 33, further comprising generating a metric threshold using the samples of the plurality of subjects, determining a metric for the sample of the patient, and characterizing the cfDNA fragments in the sample of the patient by comparing the metric with the metric threshold.
